(19) United States
(12) Patent Application Publication
(10) Pub. No.: US 2026/0083844 A1
(43) Pub. Date: Mar. 26, 2026

(54) DOWNREGULATING INOS TO INCREASE CAR-T KILLING

(71) Applicant: H. LEE MOFFITT CANCER CENTER AND RESEARCH INSTITUTE INC., TAMPA, FL (US)

(72) Inventors: Marco DAVILA, Tampa, FL (US); Sae Bom LEE, Tampa, FL (US)

(21) Appl. No.: 19/109,722

(22) PCT Filed: Sep. 15, 2023
(86) PCT No.: PCT/US2023/074366

§ 371 Date: Mar. 7, 2025

Related U.S. Application Data
(60) Provisional application No. 63/375,752, filed on Sep. 15, 2022.

(51) Int. Cl. A61K 40/11; A61K 31/155; A61K 31/198; A61K 31/381; A61K 31/416; A61K 31/4164; A61K 40/31; A61K 40/42; A61P 35/00; C07K 16/24; C12N 15/113

(52) U.S. Cl. CPC A61K 40/11; A61K 31/155; A61K 31/198; A61K 31/381; A61K 31/416; A61K 31/4164; A61K 40/31; A61K 40/4211; A61P 35/00; C07K 16/249; C12N 15/1136; C12N 2310/14

(57) ABSTRACT

Chimeric antigen receptor (CAR) T cell therapies have revolutionized the treatment of B cell malignancies, but a significant proportion of patients with large B cell lymphoma (LBCL) experience primary resistance or relapse after CAR T cell treatment. As disclosed herein, anti-inflammatory macrophages suppress CAR-T cell expansion, induce death, and reduce CAR expression. Disclosed is a method for enhancing anti-tumor efficacy of immune effector cells, such as CAR-T cells, in a subject that involves administering to the subject a nitric oxide synthase (NOS) inhibitor.

This application claims benefit of U.S. Provisional Application No. 63/375,752, filed Sep. 15, 2022, which is hereby incorporated herein by reference in its entirety.

### BACKGROUND

Surgery, radiation therapy, and chemotherapy have been the standard accepted approaches for treatment of cancers including leukemia, solid tumors, and metastases. Immunotherapy (sometimes called biological therapy, biotherapy, or biological response modifier therapy), which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed.

A major advance for anti-cancer T cell therapy is the chimeric antigen receptor (CAR), which is a single chain variable fragment (scFv) derived from an antibody fused to the signaling domains of a T cell receptor (TCR) (Davila, M. L., et al., Oncoimmunology, 2012. 1 (9): 1577-1583). The intracellular domain of a first-generation CAR includes only CD3ζ, while second-generation CARs also include co-stimulatory domains such as CD28 or 41BB. These second-generation CAR domains support highly-efficacious tumor killing in mice and led to the clinical evaluation of CAR T cell therapies in patients. The potential of CD19-targeted CAR T cells was confirmed by reports of complete remission rates of 90% for patients with B cell acute lymphoblastic leukemia (B-ALL) (Davila, M. L., et al., Sci Transl Med, 2014. 6 (224): 224ra25; Maude, S. L., et al., N Engl J Med, 2014. 371 (16): 1507-17). However, poor CAR T cell persistence and excessive T cell activation contribute to relapses and severe toxicities, respectively, and suggest a critical need to understand CAR T cell biology (Gangadhar, T. C. and R. H. Vonderheide, Nat Rev Clin Oncol, 2014. 11 (2): 91-9). Furthermore, relapses and toxicities have been seen with all second-generation CARs suggesting that the addition of co-stimulatory domains to CARs improved efficacy, but at the cost of biologic complications.

### SUMMARY

As disclosed herein, anti-inflammatory macrophages suppress CAR-T cell expansion, induce death, and reduce CAR expression. Disclosed is a method for enhancing anti-tumor efficacy of immune effector cells, such as CAR-T cells, in a subject that involves administering to the subject a nitric oxide synthase (NOS) inhibitor or interferon gamma (IFN-γ) inhibitor. In some embodiments the immune effector cell is engineered to express the NOS inhibitor or IFN-γ inhibitor. For example, the IFN-γ inhibitor can be an IFN-γ neutralizing antibody, soluble receptor, or a gene silencing oligonucleotide optionally expressed by the immune effector cell.

In some embodiments, the immune effector cells also express chimeric antigen receptor (CAR) polypeptides or transgenic TCRs, and can be used with adoptive cell transfer to target and kill cancers.

In some embodiments, the immune effector cell is selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell. In some embodiments, the immune effector cell is a CAR-T cells.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1D show macrophages in the pre-CAR T treatment TME are linked to therapeutic responses to CAR T cell therapy in LBCL patients. FIG. 1A shows bulk RNA-seq analysis on patient tumor biopsies taken before lymphodepletion conditioning therapy and axi-cel infusion (DR, 18 patients; NDR, 26 patients). Percentages of M0, M1, and M2-like macrophages based on CIBERSORTx. FIG. 1B shows progression-free survival in patients stratified according to the abundance of CIBERSORTx-defined M2-like macrophages (“Low” represents patients with a <5% M2 macrophage population, 14 patients; “High” represents patients with a >10% M2 macrophage population, 19 patients). FIGS. 1C and 1D show multiplex immunofluorescence analysis of patient tumor tissue microarray obtained prior to lymphodepletion conditioning therapy and axi-cel infusion (DR, 9 cores from 6 patients; NDR, 12 cores from 5 patients). FIGS. 1C and 1D show proportion of intratumor CD68+ macrophages and CD163+CD68+M2-like macrophages in DAPI+ cells. Data in FIGS. 1A, 1C, 1D are the mean±SEM. Statistical significance was determined by unpaired two-tailed t tests with Welch's correction (FIGS. 1A, 1C, 1D) or log-rank Mantel-Cox test (FIG. 1B). *, P<0.05; **, P<0.01; ns, not significant; DR, durable response; NDR, non-durable response.

FIGS. 2A to 2J show exposure of imMac provokes CAR T cell dysfunction. FIG. 2A is a schematic showing how CAR T cells and Eμ-myc cells were cocultured with unMac or imMac or without macrophages. FIG. 2B shows CAR T cell death at 48 h assessed by 7-aminoactinomycin D (7-AAD) incorporation via flow cytometry (n=4). FIG. 2C shows DNA replication of CAR T cells at 42 h measured by bromodeoxyuridine (BrdU) incorporation via flow cytometry (n=4). FIG. 2D shows CAR T cell expansion measured over time for 48 h by live cell analysis system (n=4). FIG. 2E shows total CAR expression levels in CAR T cells at 48 h assessed via flow cytometry (n=4). FIG. 2F shows surface CAR expression levels in CAR T cells at 48 h detected by staining single-chain variable fragments (scFv) of CAR with protein L and analyzed by flow cytometry (n=4). FIG. 2G is a schematic showing how CAR T cells were isolated after initial coculture with Eμ-myc cells with unMac or imMac or without macrophages (No Mac) for 48 h, subsequently cocultured with fresh Eμ-myc cells. FIGS. 2H and 21 show levels of IFN-γ and TNF-α in coculture supernatants of CAR T cells and Eμ-myc cells at 36 h (n=4). FIG. 2J shows lysis of Eμ-myc cells by CAR T cells assessed at 36 h using a bioluminescence assay (n=4). All data are the mean±SD. Statistical significance was determined by one-way ANOVA with Bonferroni correction for multiple comparisons. *, P<0.05; ***, P<0.001; ****, P<0.0001; ##, P<0.01; ###, P<0.001; ####, P<0.0001. BM, bone marrow; unMac, unpolarized macrophages; imMac, immunoregulatory macrophages; No Mac, without macrophages; MFI, mean fluorescence intensity; exp: exposed, isolated CAR T cells from prior coculture.

FIGS. 3A to 3H show ImMac-exposed CAR T cells upregulate iNOS. FIG. 3A is a schematic showing how CAR T cells and Eμ-myc cells were cocultured with unMac or imMac or without macrophages (No Mac) for 24 h. Coculture supernatants were analyzed by global metabolomics using LC-MS. FIGS. 3B and 3C show quantification of relative abundance of metabolites in the supernatants derived from cocultures containing imMac versus No Mac (FIG. 3B) or unMac (FIG. 3C). FIGS. 3D-3F show arginine, ornithine, and citrulline levels in the supernatants of coculture groups (n=3). The data were normalized to the mean value of CAR T and Eμ-myc cell cocultures without macrophages. FIG. 3G shows flow cytometry analysis of expression of ARG-1 and iNOS in unMac or imMac cocultured with Eμ-myc cells in the presence or absence of CAR T cells at 24 h (n=3). FIG. 3H shows nitric oxide levels in the supernatants derived from cocultures of CAR T cells and Eμ-myc cells with unMac or imMac or without macrophages at 48 h analyzed by Griess assay (n=4). Data in FIGS. 3D-3H are presented in mean±SD. Statistical significance was determined by unpaired two-tailed Student's t tests (FIG. 3B, 3C, 3G) or one-way ANOVA with Bonferroni correction for multiple comparisons (FIGS. 3D-3F, 3H). ***, P<0.001; ****, P<0.0001; ns, not significant; FC, fold change; unMac, unpolarized macrophages; imMac, immunoregulatory macrophages; No Mac, without macrophages; LC-MS, liquid chromatography-mass spectrometry.

FIGS. 4A to 4J show iNOS upregulation in imMac drives suppression of CAR T cell function. FIG. 4A shows CAR T cells and Eμ-myc cells cocultured with unMac or imMac or without macrophages in the presence or absence of L-NIL. CAR T cell expansion was measured over time for 48 h by live cell analysis system (n=4). FIGS. 4B-4D show after initial coculture of CAR T cells and Eμ-myc cells with unMac or imMac or without macrophages (No Mac) in the presence or absence of L-NIL for 48 h, isolated CAR T cells cocultured with fresh Eμ-myc cells. FIG. 4B shows luciferase-based lysis of Eμ-myc cells by CAR T cells was assessed at 24 h (n=4). FIGS. 4C and 4D show levels of IFN-γ and TNF-α in coculture supernatants at 36 h were analyzed (n=4/group). FIG. 4E shows CAR T cells and Eμ-myc cells were cocultured with WT or iNOS^{-/-} unMac or imMac or without macrophages. CAR T cell expansion was measured over time for 48 h by live cell analysis system (n=4). FIG. 4F shows after initial coculture of CAR T cells and Eμ-myc cells with WT or iNOS-unMac or imMac or without macrophages (No Mac) for 48 h, isolated CAR T cells were cocultured with fresh Eμ-myc cells. Luciferase-based lysis of Eμ-myc cells by CAR T cells was assessed at 36 h (n=4). FIGS. 4G and 4H shows CAR T cells cocultured with Eμ-myc cells in the presence or absence of NCX-4016 or PNT. CAR T cell expansion was measured over time for 48 h by live cell analysis system (n=3-4). FIG. 4I shows after initial coculture of CAR T cells and Eμ-myc cells in the presence or absence of NCX-4016 (100 UM) or PNT (50 UM) for 48 h, isolated CAR T cells were cocultured with fresh Eμ-myc cells. Luciferase-based lysis of Eμ-myc cells by CAR T cells was assessed at 24 h (n=4). FIG. 4J shows CAR T cells and Eμ-myc cells cocultured with or without imMac in the presence or absence of c-PTIO. CAR T cell expansion was measured over time for 48 h by live cell analysis system (n=3). All data are presented in mean±SD. Statistical significance was determined by unpaired two-tailed Student's t tests (FIGS. 4A, 4B, and 4E-4J) or one-way ANOVA with Bonferroni correction for multiple comparisons (FIGS. 4C, 4D). *, P<0.05; **, P<0.01; ***, P<0.001; ****, P<0.0001; ####, P<0.0001; PNT, peroxynitrate; c-PTIO, carboxyl-PTIO; unMac, unpolarized macrophages; imMac, immunoregulatory macrophages; No Mac, without macrophages; exp: exposed, isolated CAR T from prior coculture.

FIGS. 5A to 5F show CAR T cell-derived IFN-γ induces iNOS in imMac. FIGS. 5A-5C show CAR T cells and Eμ-myc cells cocultured with unMac or imMac in the presence of anti-IFN-γ or IsoCon. FIG. 5A shows expression of ARG-1 and iNOS in unMac or imMac was analyzed at 24 h via flow cytometry (n=3). FIG. 5B shows nitric oxide levels in coculture supernatants at 44 h were analyzed by Griess assay (n=4). FIG. 5C shows CAR T cell expansion was measured over time for 48 h by live cell analysis system (n=4). FIGS. 5D-5F shows after initial coculture of CAR T cells and Eμ-myc cells with unMac or imMac in the presence of Isotype control (IsoCon) or anti-IFN-γ for 48 h, isolated CAR T cells were cocultured with fresh Eμ-myc cells. FIG. 5D shows luciferase-based lysis of Eμ-myc cells by CAR T cells assessed at 24 h (n=4). FIGS. 5E and 5F show levels of IFN-γ and TNF-α in the coculture supernatants at 24 h analyzed (n=4). All data are presented in mean±SD. Statistical significance was determined by unpaired two-tailed Student's t tests (FIGS. 5A, 5C, 5D) or one-way ANOVA with Bonferroni correction for multiple comparisons (FIGS. 5B, 5E, 5F). *, P<0.05; **, P<0.01; ***, P<0.001; ****, P<0.0001; IsoCon, isotype control; unMac, unpolarized macrophages; imMac, immunoregulatory macrophages; exp: exposed, isolated CAR T from prior coculture.

FIGS. 6A to 6M show iNOS-expressing imMac induces CAR T cell metabolic dysregulation. FIGS. 6A-6K show CAR T cells and Eμ-myc cells cocultured with unMac or imMac or without macrophages (No Mac) in the presence or absence of L-NIL. After coculture for 48 h, global metabolomics was performed on CAR T cells. Quantification of relative abundance of metabolites in CAR T cells derived from cocultures with imMac versus No Mac (FIG. 6A) or unMac (FIG. 6B). FIG. 6C is a schematic depicting altered metabolites associated with glycolysis pathway and TCA cycle. FIGS. 6D-6K show levels of F1,6BP, G3P, DHAP, citrate, aconitate, succinate, malate, and itaconate in coculture groups (n=4). The data were normalized to the mean value of CAR T cells derived from cocultures without macrophages and L-NIL treatment. FIG. 6L shows CAR T cells cocultured with Eμ-myc cells in the presence or absence of 4-OI. CAR T cell expansion measured over time for 48 h (n=3-4). FIG. 6M shows CAR T cells and Eμ-myc cells cocultured with imMac or without macrophages (No Mac) in the presence or absence of L-NIL. After coculture for 48 h, seahorse assay was performed on CAR T cells (n=6). ECAR was measured in response to glucose (Glc), ATP synthase inhibitor (Oligo), or hexokinase II inhibitor (2-DG). OCR was measured in response to Oligo, mitochondrial oxidative phosphorylation uncoupler (FCCP), or electron transport chain complex I/III inhibitor (Rot/AA). Data in (D-K) are presented in mean±SD. Statistical significance was determined by unpaired two-tailed Student's t tests (A, B, L) or one-way ANOVA with Bonferroni correction for multiple comparisons (D-K). *, P<0.05; **, P<0.01; ***, P<0.001; ****, P<0.0001; ns, not significant; F1,6BP, 1,6-bisphosphate; G3P, glyceraldehyde 3-phosphate; DHAP, dihydroxyacetone phosphate; 4-OI, 4-octyl itaconate; ECAR, extracellular acidification rate; OCR, oxygen consumption rate; Oligo, oligomycin A; 2-DG, 2-deoxy-D-glucose; FCCP, carbonyl cyanide p-trifluoromethoxyphenylhydrazone; Rot/AA, rotenone, antimycin A; unMac, unpolarized macrophages; imMac, immunoregulatory macrophages; exp: exposed, isolated CAR T from prior coculture.

FIGS. 7A to 7H show iNOS inhibition improves efficacy of CAR T cell therapy. FIG. 7A shows experimental settings for FIGS. 7B-7E where Eμ-myc cells were intraperitoneally injected into Rag1^{-/-} mice. Seven days later, WT 19dz or WT 1928z or IFN-γ^{-/-} 1928z CAR T cells were transferred into mice. Peritoneal lavage cells were obtained 24-40 h after CAR T cell transfer. FIGS. 7B-7D show flow cytometry analysis of iNOS+ cells among CD11b+F4/80+ macrophages (FIG. 7B), ARG-1+ cells among CD11b+F4/80+ macrophages (FIG. 7C), and F4/80+ macrophages among CD45+ cells (FIG. 7D) at 24 h after CAR T cell transfer (n=6/group). FIG. 7E shows CD11b+ peritoneal myeloid cells obtained 40 h following WT 1928z CAR T cell transfer (n=5 mice). CD11b+ cells were then ex vivo cocultured with fresh CAR T cells and Eμ-myc cells in the presence or absence of L-NIL (coculture ratio, 1:1:1=CD11b+ cell:CAR T:Eμ-myc cell). Expansion of CAR T cells was measured over time for 48 h by live cell analysis system (n=4-5). FIG. 7F shows experimental settings for FIG. 7G. FIG. 7G shows percentage of survival of tumor-bearing mice treated with WT 19dz or WT 1928z CAR T cells receiving L-NIL or PBS (vehicle). Results are from two pooled independent experiments (PBS, n=12 mice; L-NIL, n=12 mice; 19dz CAR T+PBS, n=11 mice; 19dz CAR T+L-NIL, n=12 mice; 1928z+PBS, n=28 mice; 1928z+L-NIL, n=34 mice). FIG. 7H shows multiplex immunofluorescence analysis of patient tumor tissue microarray obtained prior to lymphodepletion conditioning therapy and axi-cel infusion (DR, 9 cores from 6 patients; NDR, 12 cores from 5 patients). Proportion of intratumor iNOS+CD14+ cells in DAPI+ cells. Data in FIGS. 7B, 7C, 7D are presented in mean±SEM and FIG. 7E are presented in mean±SD. Statistical significance was determined by one-way ANOVA with Bonferroni correction for multiple comparisons (FIG. 7B, 7C, 7D), unpaired two-tailed Student's t tests (FIG. 7E), log-rank Mantel-Cox test (FIG. 7G), or unpaired two-tailed t tests with Welch's correction (FIG. 7H). *, P<0.05; **, P<0.01; ***, P<0.001.

FIGS. 8A to 8E show immune cell composition of pre-CAR T treatment TME. FIGS. 8A and 8B show patient tumor biopsies were taken prior to lymphodepletion conditioning therapy and axi-cel infusion in LBCL patients (DR, 18 patients; NDR, 26 patients). FIG. 8A is a heatmap showing relative abundances of CIBERSORTx deconvoluted immune cell types. FIGS. 8B-8E show multiplex immunofluorescence analysis of patient tumor tissue microarray obtained prior to lymphodepletion conditioning therapy and axi-cel infusion (DR, 9 cores from 6 patients; NDR, 12 cores from 5 patients). Proportion of intratumor CD3+, CD4+CD3+, CD4-CD3+, and Foxp3+CD4+ T cells in DAPI+ cells. Data in FIGS. 8B-8E are the mean±SEM. Statistical significance was determined by unpaired two-tailed t tests with Welch's correction. ns, not significant; DR, durable response; NDR, non-durable response.

FIG. 9 shows exposure of imMac suppresses CAR T cell expansion. Construct maps encoding anti-CD19 CAR. All included a 5′ long terminal repeat (LTR), anti-murine CD19 single-chain variable fragment (scFv), CD8 transmembrane and hinge domain (CD8 TM), glycine-serine linker (G/S), mCherry, and 3′ LTR. 19dz CAR is 1st generation CAR containing truncated CD3z chain. 1928z CAR is 2nd generation CAR containing CD28 costimulatory domain and CD3z chain.

FIGS. 10A to 10C show exposure to CAR T cells induces phenotypic changes in macrophages. FIG. 10A shows arginine metabolic pathways. FIG. 10B shows CAR T cells and Eμ-myc cells cocultured with unMac or imMac for 24 h. Expression of ARG-1 and iNOS in CD3+ T cells or CD19+Eμ-myc cells were analyzed by flow cytometry. FIG. 10C shows flow cytometry analysis of expression of PD-L1 in unMac or imMac cocultured with Eμ-myc cells in the presence or absence of CAR T cells at 24 h (n=3). Data in FIG. 10C are presented in mean±SD. Statistical significance was determined by unpaired two-tailed Student's t tests. ****, P<0.0001. ARG-1, arginase-1; iNOS, inducible nitric oxide synthase; NO, nitric oxide.

FIGS. 11A to 11L show arginine metabolism by imMac mediates impairment of CAR T cell function. FIG. 11A shows arginine metabolic pathway and the inhibitors of respective enzymes. FIGS. 11B and 11C show CAR T cells and Eμ-myc cells cocultured with unMac or imMac or without macrophages in the culture media containing supraphysiological (1.15 mM) (FIG. 11B) or physiological (150 UM) (FIG. 11C) concentration of arginine in the presence or absence of nor-NOHA. CAR T cell expansion was measured over time for 48 h by live cell analysis system (n=4). FIG. 11D shows flow cytometry analysis of expression of ARG-1 and PD-L1 in WT or iNOS^{-/-} imMac cocultured with CAR T cells and Eμ-myc at 24 h. FIG. 11E shows analysis of nitric oxide levels in coculture supernatants of CAR T cells and Eμ-myc cells with or without WT or iNOS-unMac or imMac in the presence or absence of L-NIL at 48 h using Griess assay (n=4). FIG. 11F shows analysis of nitric oxide levels in coculture supernatants of CAR T cells and Eμ-myc cells with or without WT or iNOS^{-/-} unMac or imMac at 48 h using Griess assay (n=4). FIGS. 11G-11I show citrulline, arginine, and ornithine levels in coculture supernatants of CAR T cells and Eμ-myc cells with or without WT or iNOS^{-/-} unMac or imMac in the presence or absence of L-NIL (n=3). Graphs contain control data also used in FIGS. 3D-3F. FIG. 11J shows CAR T cells and Eμ-myc cells cocultured in the increasing concentrations of citrulline. CAR T cell expansion was measured over time for 48 h by live cell analysis system (n=4). FIGS. 11K and 11L show after initial coculture of CAR T cells and Eμ-myc cells in the presence or absence of NCX-4016 (100 UM) or PNT (50 μM) for 48 h, isolated CAR T cells were cocultured with fresh Eμ-myc cells. Levels of IFN-γ and TNF-α in coculture supernatants at 24 h were measured (n=4). All data are presented in mean±SD. Data in FIG. 11E, 11F, 11G-11I, 11K, 11L, statistical significance was determined by one-way ANOVA with Bonferroni correction for multiple comparisons. **, P<0.01; ****, P<0.0001; ns, not significant.

FIGS. 12A to 12C show IFN-γ^{-/-} CAR T cells do not trigger iNOS in macrophages. WT or IFN-γ^{-/-} CAR T cells were cocultured with Eμ-myc cells with unMac or imMac. FIG. 12A shows expression of ARG-1 and iNOS in unMac or imMac at 24 h analyzed by flow cytometry (n=3). FIG. 12B shows nitric oxide levels in coculture supernatants at 48 h were analyzed by Griess assay (n=4). FIG. 12C shows CAR T cell expansion measured over time for 48 h by live cell analysis system (n=4). All data are presented in mean±SD. Statistical significance was determined by unpaired two-tailed Student's t tests (FIG. 12A, 12C) or one-way ANOVA with Bonferroni correction for multiple comparisons (FIG. 12B). ***, P<0.001; ****, P<0.0001.

FIGS. 13A to 13G show itaconate is endogenously produced in imMac-exposed CAR T cells. CAR T cells and Eμ-myc cells were cocultured with unMac or imMac or without macrophages in the presence or absence of L-NIL for 48 h. FIGS. 13A-13G show ¹³C6-glucose tracing performed on isolated CAR T cells. FIG. 13A is a schematic showing ¹³C-labeled TCA cycle-related metabolites generated from ¹³C₆-glucose. Colored circles represent ¹³C-labeled carbons. FIGS. 13B-13G show levels of ¹³C-labeled citrate, aconitate, itaconate, α-ketoglutarate, fumarate, and malate in coculture groups (n=3). FIG. 13H shows immunoblotting performed on isolated CAR T cells. The β-actin was used as a loading control. Data in FIGS. 13B-13G are presented in mean±SD. Statistical significance was determined by one-way ANOVA with Bonferroni correction for multiple comparisons. *, P<0.05; **, P<0.01; ***, P<0.001; ****, P<0.0001; IDH, isocitrate dehygrogenase; IRG1, immune response gene 1; unMac, unpolarized macrophages; imMac, immunoregulatory macrophages; exp: exposed, isolated CAR T from prior coculture.

FIG. 14 shows iNOS+myeloid cells in the TME are associated with poor therapeutic responses to CAR T cell therapy. Flow cytometry gating strategy for the identification of TAM expression of ARG-1 and iNOS in Eμ-myc tumors.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “protein domain” refers to a portion of a protein, portions of a protein, or an entire protein showing structural integrity; this determination may be based on amino acid composition of a portion of a protein, portions of a protein, or the entire protein.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

### Chimeric Antigen Receptors (CAR)

CARs generally incorporate an antigen recognition domain from the single-chain variable fragments (scFv) of a monoclonal antibody (mAb) with transmembrane and intracellular signaling motifs involved in lymphocyte activation (Sadelain M, et al. Nat Rev Cancer 2003 3:35-45). A CAR is generally made up of three domains: an ectodomain, a transmembrane domain, and an endodomain. The ectodomain comprises the antigen-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

Additional CAR constructs are described, for example, in Fresnak A D, et al. Engineered T cells: the promise and challenges of cancer immunotherapy. Nat Rev Cancer. 2016 Aug. 23; 16 (9): 566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD33 domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. SCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3Z chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3Z signaling domain by itself or combined with any other desired cytoplasmic domain(s) useful in the context of the CAR of the invention. For example, the cytoplasmic domain of the CAR can comprise a CD3Z chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4 (2): 89-99 (2004)). The hinge sequence may be positioned between the antigen recognition moiety (e.g., anti-CD123 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the antigen binding agent is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the antigen binding agent is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two tumor antigens. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

### Transgenic T-Cell Receptor (TCR)

In some embodiments, the immune effector cells also express a transgenic TCRs, and can be used with adoptive cell transfer to target and kill cancers. The T-cell receptor (TCR) is a molecule found on the surface of T cells which is responsible for recognizing fragments of target antigen as peptides bound to major histocompatibility complex (MHC) molecules. The TCR is a heterodimer composed of two different protein chains. In humans, in 95% of T cells the TCR consists of an alpha (a) chain and a beta (b) chain (encoded by TRA and TRB, respectively), whereas in 5% of T cells the TCR consists of gamma and delta (g/d) chains (encoded by TRG and TRD, respectively).

Each chain is composed of two extracellular domains: a variable (V) region and a constant (C) region. The constant region is proximal to the cell membrane, followed by a transmembrane region and a short cytoplasmic tail, while the variable region binds to the peptide/MHC complex. The variable domain of the TCR α-chain and b-chain each have three hypervariable or complementarity determining regions (CDRs). CDR3 is the main CDR responsible for recognizing processed antigen. The constant domain of the TCR consists of short connecting sequences in which a cysteine residue forms disulfide bonds, which form a link between the two chains. When the TCR engages with antigenic peptide and MHC (peptide/MHC), the T lymphocyte is activated through signal transduction. In contrast to conventional antibody-directed target antigens, antigens recognized by the TCR can include the entire array of potential intracellular proteins, which are processed and delivered to the cell surface as a peptide/MHC complex. It is possible to engineer cells to express heterologous (i.e. non-native) TCR molecules by artificially introducing the TRA and TRB genes; or TRG and TRD genes into the cell using a vector. Such heterologous TCRs may also be referred to herein as transgenic TCRs. For example, the genes for genetically modified TCRs may be reintroduced into autologous T cells and transferred back into patients for T cell adoptive therapies. In some embodiments, the transgenic TCR is a recombinant TCR.

### Tumor Antigens

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG16, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Inhibitors

Disclosed is a method for enhancing anti-tumor efficacy of immune effector cells, such as CAR-T cells, in a subject that involves administering to the subject a nitric oxide synthase (NOS) inhibitor or interferon gamma (IFN-γ) inhibitor.

Exemplary iNOS-inhibitory compounds include, without limitation, NG-monomethyl-L-arginine [L-NMMA], (N-[[3-(aminomethyl)phenyl]methyl]-ethanimidamide) [1400 W], (N5-[imino (nitroamino) methyl]-L-ornithine methyl ester) [L-NAME; C7H15N504.HCl; MW=269.69; CAS 51298-62-5], as well as salts, derivatives, and combinations thereof. Additional NOS inhibitors are described in Table 1.

TABLE 1
NOS inhibitors

| NOS | | Speci- |
|---|---|---|
| inhibitor | Full chemical name | ficity |
| L-NAME | NG-Nitro-L-arginine-methyl ester HCl | NOS |
| L-NMMA | NG-Monomethyl-L-arginine monoacetate | NOS |
| S-Ethyl ITU | S-Ethylisothiourea HBr | NOS |
| 7-NI | 7-Nitroindazole | NOS1 |
| 3-Bromo-7-NI | 3-Bromo-7-nitroindazole | NOS1 |
| Vinyl-L-NIO | N5-(1-Imino-3-butenyl)-L-ornithine | NOS1 |
| AR-R17477 | N-(4-(2-((3- | NOS1 |
| | Chlorophenylmethyl)amino)ethyl)phenyl)-2- | |
| | thiophecarboxamidine dihydrochloride | |
| NAAANG | (4S)-N-(4-Amino-5[aminoethyl]aminopentyl)- | NOS1 |
| | N′-nitroguanidine 3TFA | |
| ETPI | S-Ethyl-N-[4- | NOS1 |
| | (trifluoromethyl)phenyl]isothiourea HCl | |
| TRIM | 1-(2-Trifluoromethylphenyl)imidazole | NOS1 |
| N-Propyl-L- | N5-[Imino(propylamino)methyl]-L-ornithine | NOS1 |
| arginine | | |
| L-NNA | NG-Nitro-L-arginine | NOS1 |
| 1400W | N-(3-(Aminomethyl)benzyl)acetamidine 2HCl | NOS2 |
| L-NIL | L-N6-(1-Iminoethyl)-lysine | NOS2 |
| AMT | 2-Amino-5,6-dihydro-6-methyl-4H-1.3-thiazine | NOS2 |
| | HCl | |
| 1,3 PB-ITU | S,S′-(1,3-Phenylene-bis(1,2-ethanediyl))bis- | NOS2 |
| | isothiourea 2HBr | |
| 1,4 PB-ITU | S,S′-(1,4-Phenylene-bis(1,2-ethanediyl))bis- | NOS2 |
| | isothiourea 2HBr | |
| GW274150 | (S)-2-Amino-(1-iminoethylamino)-5- | NOS2 |
| | thioheptanoic acid | |
| GED | Guanidinoethyldisulfide 2H2CO3 | NOS2 |
| MEG | Mercaptoethylguanidine sodium succinate | NOS2 |
| S-Methyl ITU | S-methylisothiourea sulfate | NOS2 |
| S-Aminopropyl | S-(3-Aminopropyl)isothiourea 2HBr | NOS2 |
| ITU | | |

Therefore, in some embodiments, the inhibitor is a compound having the chemical structure:

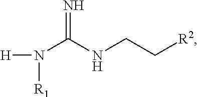

wherein R1 represents a C1-C6 alkyl, C2-C6 alkenyl, C2-C6 alkynyl, OH, H, —NO2, or halide group; and R2 represents a substituted or unsubstituted C1-C30, straight chain or branched alkyl group, wherein the alkyl, if substituted, is substituted by a hydroxy, carboxy, amino, acetoxy, or nitroxy group or a pharmaceutically acceptable salt or ester thereof.

In some embodiments, the inhibitor is guanidinoethyldisulfide (GED) which has the structure:

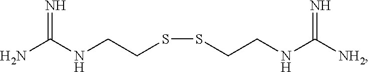 [SMILES: N=C(N)NCCSSCCNC(=N)N]

\or a pharmaceutically acceptable salt or ester thereof.

In some embodiments, the inhibitor is nitro-L-arginine methyl ester (L-NAME) which has the structure:

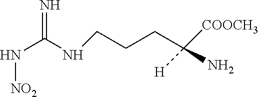 [SMILES: COC(=O)[C@@H](N)CCCNC(=N)N[N+](=O)[O-]]

or a pharmaceutically acceptable salt or ester thereof.

In some embodiments, the inhibitor is N-monomethyl-L-arginine (L-NMMA) which has the structure:

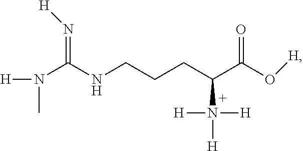

or a pharmaceutically acceptable salt or ester thereof.

In some embodiments, the IFN-γ inhibitor is an IFN-γ neutralizing antibody. Anti-IFN-γ antibodies blocking various biological activities of native IFN-γ (often referred to as neutralizing antibodies”) are known in the art, and are, for example, disclosed in the following publications: Billiau, A., Immunol. Today 9, 37-40 (1988); Hereman, H. et al., J. Exp. Med. 171, 1853-1859 (1990); Landolfo, S. et al., Science 229, 176-179 (1985); Didlake, R. H. et al., Transplantation 45, 222-223 (1988), Jacob, C. O. et al., J. Exp. Med. 166, 789-803 (1987); Yong, V. W. et al., Natl. Acad. Sci. USA 88, 7016-7020 (1991)]. Antibodies to a native IFN-γ receptor which inhibit the binding of native IFN-γ to its receptor and thereby block IFN-γ biological activity are, for example, disclosed in EP 369,413; EP 393,502; EP 416,652; EP 240,975; and U.S. Pat. No. 4,897,264, which are incorporated by references for the teaching of these inhibitors.

In some embodiments, the IFN-γ inhibitor is an inactive IFN-γ variant. The recombinant production of IFN-γ was first reported by Gray, Goeddel and co-workers [Gray et al., Nature 295, 503-508 (1982)], and is subject of U.S. Pat. Nos. 4,762,791, 4,929,544, 4,727,138 and 4,925,793. Recombinant IFN-γ polypeptides lacking the first three N-terminal amino acids (CysTyrCys), including variously truncated derivatives are, for example, disclosed in European Publication No. 146,354. Non-human animal interferons, including IFN-γ, are, for example, disclosed in European Publication No. 88,622.

In some embodiments, the IFN-γ inhibitor is an extracellular domain of an IFN-γ receptor, optionally fused to a stable plasma protein. IFN-γ receptors purified from native source or produced by techniques of recombinant DNA technology are known in the art, and are, for example disclosed in the following publications: Aguet, M. & Merlin, G., J. Exp. Med. 165, 988-999 (1987); Novick, D. et al., J. Biol. Chem. 262, 8483-8487 (1987); Calderon, J. et al., Proc. Natl. Acad. Sci. USA 85, 4837-4841 (1988); Basu, M. et al., Proc. Natl. Acad. Sci. USA 85, 6282-6286 (1988); Aguet et al., Cell 55, 273-280 (1988); Gray, P. W. et al., Proc. Nat. Acad. Sci. USA 86, 8497-8501 (1989).

In some embodiments, the IFN-γ inhibitor is a gene silencing oligonucleotide. Gene silencing oligonucleotide, but are not limited to, antisense DNA oligonucleotides, miRNA, gRNA, siRNA, shRNA, or other RNAi molecules. In one embodiment, the nucleic acid is an siRNA for targeting a gene expression product.

Typically, the inhibitor is administered as a component of a composition which comprises a pharmaceutically acceptable carrier. In some embodiments, the pharmaceutically acceptable carrier comprises a biopolymer, such as hyaluronic acid or a pharmaceutically acceptable salt or ester thereof, e.g. sodium hyaluronate.

In some embodiments, the inhibitor is present within the composition at a concentration between 0.1% and 5.0% by weight, such as between 0.1% and 1.0% by weight. In some embodiments, the amount of the inhibitor administered to the subject is between 0.1 mg/kg and 30 mg/kg body weight of the subject. For certain inhibitors the amount can be between 0.3 mg/kg and 10 mg/kg body weight of the subject and the concentration of the inhibitor is between 0.1 to 1000 micromolar, such as between 1.0 to 100 micromolar. For some inhibitors, the amount of the inhibitor administered to the subject is between 1.0 mg/kg and 30 mg/kg body weight of the subject, such as between 1.0 mg/kg and 10 mg/kg body weight of the subject, and the concentration of the inhibitor is between 1.0 to 1000 micromolar, such as between 5.0 to 100 micromolar.

### Immune Effector Cells

The disclosed immune effector cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56+CD3-large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

In some embodiments, the immune effector cells are tumor infiltrating lymphocytes (TILs) isolated from the subject and expanded ex vivo. In adoptive T cell transfer therapy, TILs are expanded ex vivo from surgically resected tumors that have been cut into small fragments or from single cell suspensions isolated from the tumor fragments. Multiple individual cultures are established, grown separately and assayed for specific tumor recognition. TILs can be expanded over the course of a few weeks with a high dose of IL-2 in 24-well plates. Selected TIL lines that presented best tumor reactivity can then be further expanded in a “rapid expansion protocol” (REP), which uses anti-CD3 activation for a typical period of two weeks. The final post-REP can be infused back into the patient. The process can also involve a preliminary chemotherapy regimen to deplete endogenous lymphocytes in order to provide the adoptively transferred TILs with enough access to surround the tumor sites.

In some embodiments, the “immune effector cells” are progenitor cells or stem cells with the potential to become lymphocytes. For example, in some embodiments, the cells are induced pluripotent stem cells (iPSCs). iPSCs are typically derived by introducing products of specific sets of pluripotency-associated genes, or “reprogramming factors”, into a given cell type. The original set of reprogramming factors (also dubbed Yamanaka factors) are the transcription factors Oct4 (Pou5f1), Sox2, cMyc, and Klf4. While this combination is most conventional in producing iPSCs, each of the factors can be functionally replaced by related transcription factors, miRNAs, small molecules, or even non-related genes such as lineage specifiers.

### Therapeutic Methods

Immune effector cells expressing the disclosed transcription factors and/or dominant negative constructs can elicit an anti-tumor immune response against tumors. The anti-tumor immune response elicited by the disclosed recombinant immune effector cells may be an active or a passive immune response. In addition, the immune response may be part of an adoptive immunotherapy approach in which recombinant immune effector cells induce an enhanced immune response.

Adoptive transfer of immune effector cells is a promising anti-cancer therapeutic. Following the collection of a patient's immune effector cells, the cells may be genetically engineered to express the disclosed transcription factors and/or dominant negative constructs, and optionally CARs, then infused back into the patient.

The disclosed recombinant immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed recombinant immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cyclophosphamide, fludarabine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of co-signaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PD-L1 inhibitor comprises an antibody that specifically binds PD-L1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, dacarbazine, hydroxyurea, asparaginase, gemcitabine or cladribine.

In some embodiments, such an additional therapeutic agent may be selected from an alkylating agent, such as mechlorethamine, thiotepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitidine.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

In some embodiments, the disclosed cells are administered in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, the disclosed cells are administered in combination with an anti-cancer cytokine, chemokine, or combination thereof. Examples of suitable cytokines and growth factors include IFNγ, IL-2, IL-4, IL-6, IL-7, IL-10, IL-12, IL-13, IL-15, IL-18, IL-23, IL-24, IL-27, IL-28a, IL-28b, IL-29, KGF, IFNα (e.g., INFa2b), GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C-C chemokine families. Suitable cytokines include cytokine derivatives, cytokine variants, cytokine fragments, and cytokine fusion proteins.

In some embodiments, the disclosed cells are administered in combination with a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, the disclosed cells are administered in combination with a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogen (such as flutamide/eulexin), a progestin (such as hydroxyprogesterone caproate, medroxy-progesterone/provera, megestrol acetate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrozole/arimidex, aminoglutethimide/cytadren, exemestane) or a hormone inhibitor (such as octreotide/sandostatin).

In some embodiments, the disclosed cells are administered in combination with an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed cells are administered in combination with surgery.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Results

### Macrophages in the Pre-CAR T Treatment TME are Linked to Therapeutic Responses in LBCL Patients

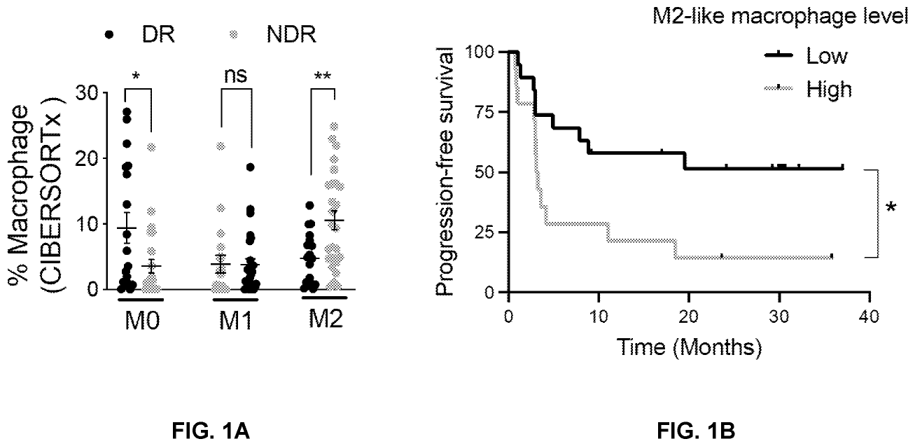

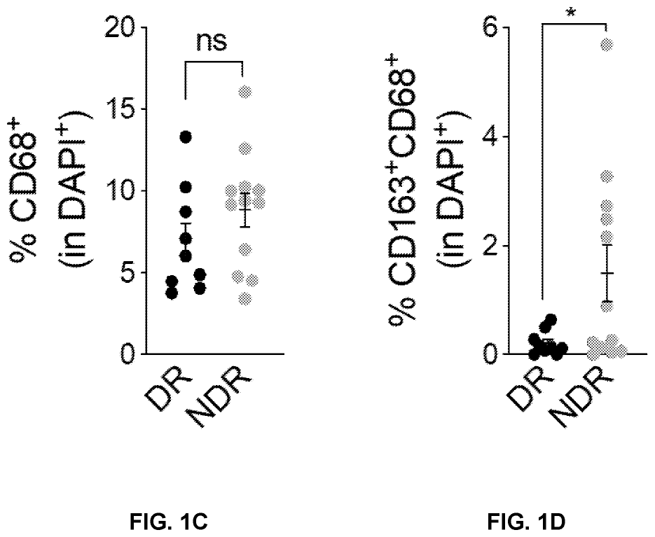

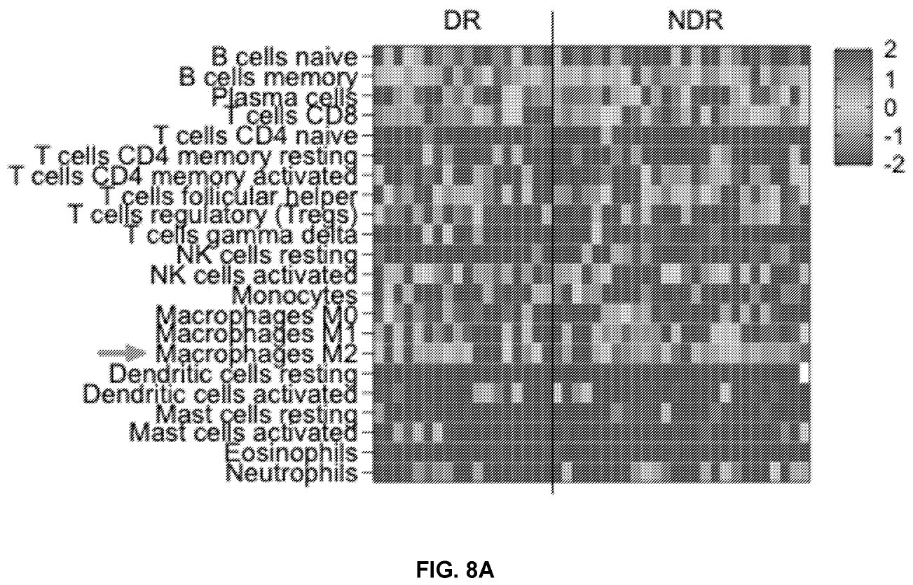

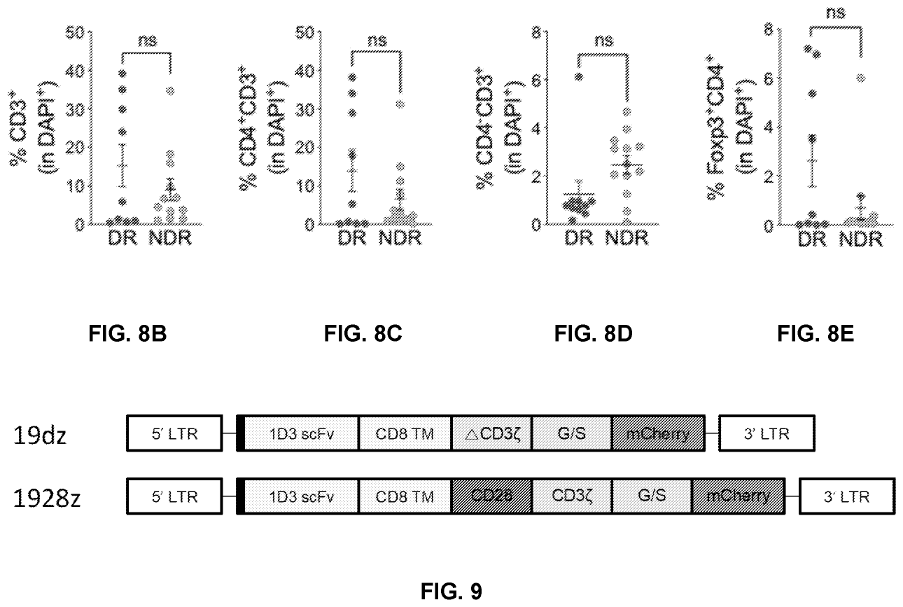

The tumor immune infiltrate and its relationship to clinical outcomes was examined in patients with LBCL receiving Axicabtagene ciloleucel (axi-cel). Bulk RNA sequencing (RNA-seq) was performed on patient tumor biopsies taken prior to lymphodepletion and CAR T cell treatment. Subsequently, CIBERSORTx was used to deconvolute intratumoral immune cell composition (FIG. 8A) (Newman et al., Nat Methods 2015 12:453-457; Newman et al., Nat Biotechnol 2019 37:773-782). Patients with non-durable responses (NDR) to CAR T cell therapy, who experienced lymphoma relapse or death by any cause, exhibited a higher proportion of transcriptionally identified M2-like macrophages compared to patients with durable responses (DR), who remained in remission for at least 6 months following axi-cel infusion (FIG. 1A). Using gene set enrichment analysis (GSEA), enrichment of M2-associated genes was identified in patients with NDR. The proportion of nonactivated macrophages (M0) was lower in patients with NDR, while levels of M1-like macrophages were similar between patients with NDR and DR (FIG. 1A). There was also a higher abundance of M2-like macrophages in patients with worse progression-free survival after axi-cel therapy (FIG. 1B).

Next, multiplex immunofluorescence imaging was used to validate the proportions of tumor-associated immune populations in tissue microarrays composed of patient tumor specimens prior to lymphodepletion and CAR T cell therapy. It was determined that patients with NDR had a similar proportion of total macrophages (CD68⁺) (FIG. 1C) but a significantly higher proportion of M2-like macrophages (CD163⁺ CD68⁺) compared to patients with DR (FIG. 1D). Notably, proportions of total T cells (CD3⁺), CD4⁺ CD3⁺ T cells, CD4⁻ CD3⁺ T cells, and Tregs (FOXP3⁺CD3⁺) did not significantly correlate with NDR or DR status (FIG. 8B-8E). These findings collectively indicate that the presence of M2-like TAMs within the TME prior to CAR T therapy is associated with poor therapeutic responses to axi-cel in patients with LBCL.

### Immunoregulatory Actions of Macrophages on CAR T Cells

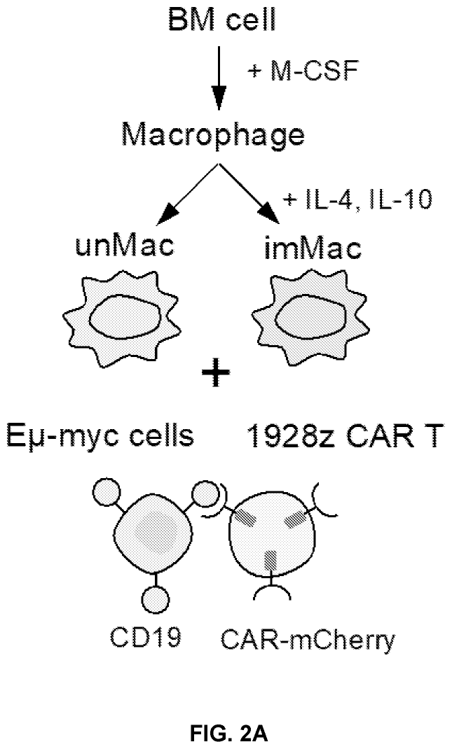

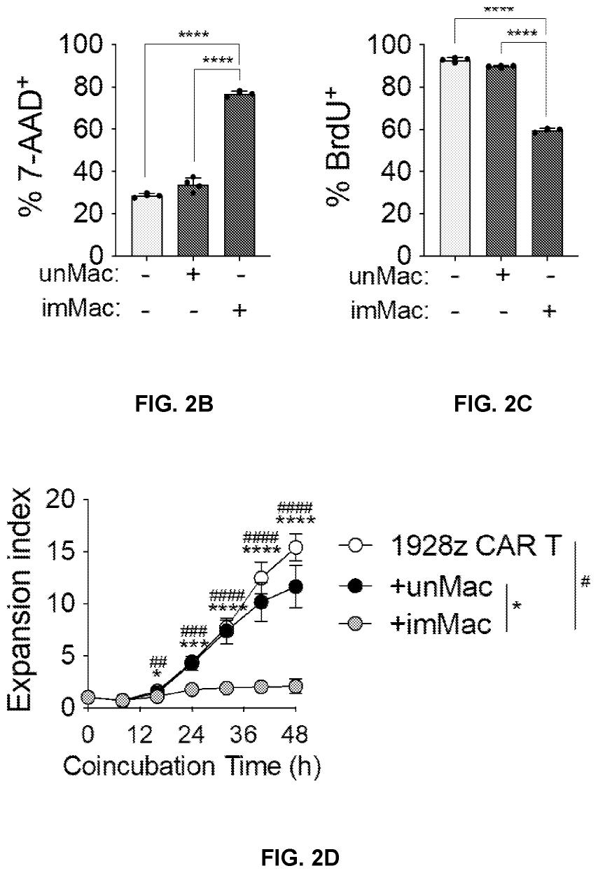

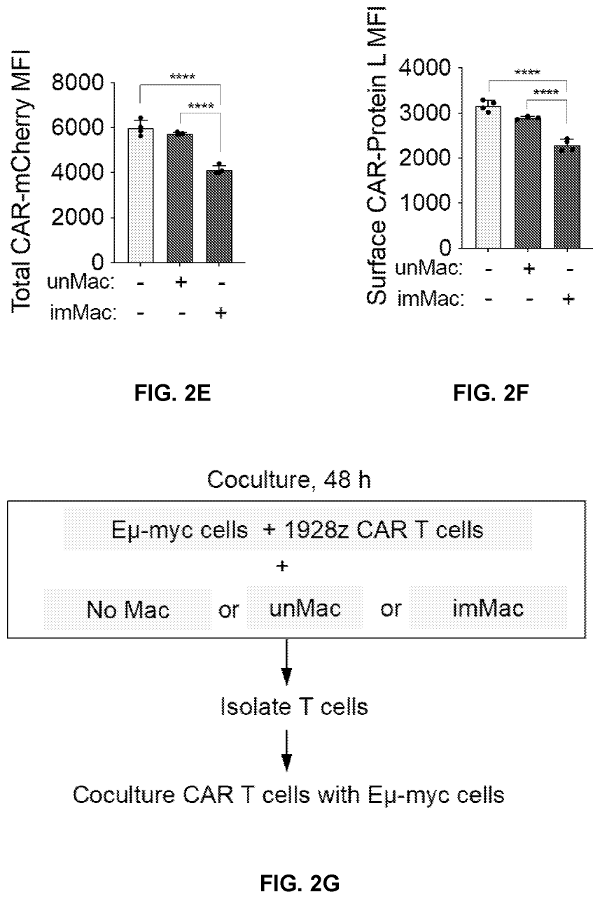

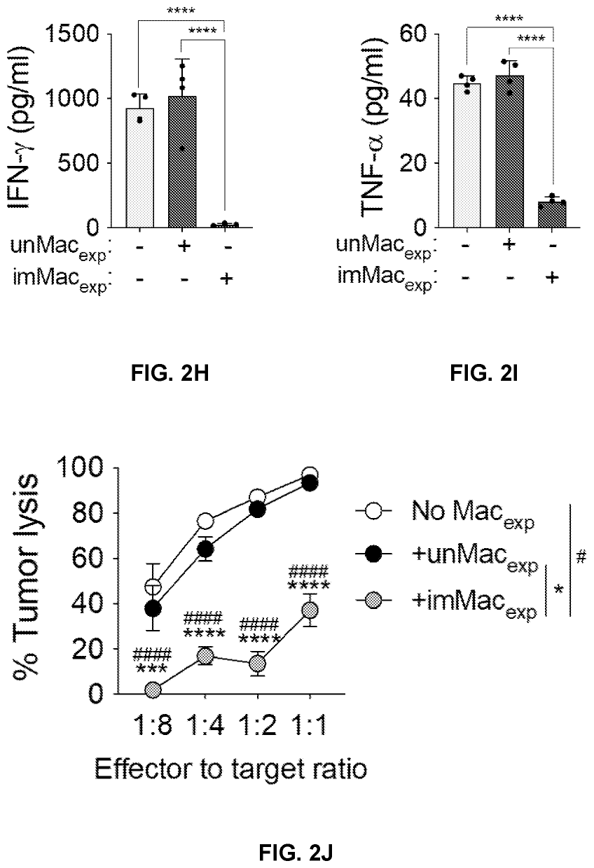

To explore how M2-like macrophages may impact the cellular function of CAR T cells, a syngeneic coculture system was employed (FIG. 2A). In this model, murine anti-CD19 CAR T cells, which include CD28 and CD3z signaling domains linked to a fluorescent mCherry reporter (1928z) (Boucher et al., Cancer Immunol Res 2021 9:62-74) (FIG. 9), were cocultured with a murine malignant B cell line (Eμ-myc cells) in the presence or absence of mouse bone marrow-derived macrophages (BMDMs). The BMDMs were differentiated into M2-like macrophages (imMac) by culturing with IL-4 and IL-10 or with media alone without further differentiation to utilize as unpolarized macrophage controls (unMac). The mCherry reporter served as a marker for CAR transduction and CAR T cell expansion.

CAR T cells cocultured with imMac showed increased cell death (FIG. 2B) and reduced DNA replication (FIG. 2C) compared to CAR T cells cocultured with unMac or without macrophages. Correspondingly, CAR T cells exhibited diminished expansion during coculture with imMac (FIG. 2D). Moreover, CAR T cells cocultured with imMac showed lower total CAR expression (FIG. 2E) as well as reduced surface CAR expression (FIG. 2F). The impact of imMac on CAR T cell effector function was next explored. To exclude the direct contribution of macrophage effector activities in these functional assays, CAR T cells, Eμ-myc cells, and macrophages were first cocultured for 48 hours (FIG. 2G). Next, CAR T cells isolated from the respective cocultures were evaluated for their effector function against fresh Eμ-myc cells. CAR T cells derived from cocultures with imMac exhibited impaired production of effector cytokines IFN-γ and tumor necrosis factor-alpha (TNF-α) (FIGS. 2H and I) and demonstrated decreased ability to lyse target tumor cells (FIG. 2J). Collectively, these results show that macrophages polarized towards an M2-like phenotype exert immunoregulatory actions that impair multiple aspects of CAR T cell biology, including survival, expansion, and CAR-dependent effector functions.

### ImMac-Exposed CAR T Cells Upregulate Inducible Nitric Oxide Synthase

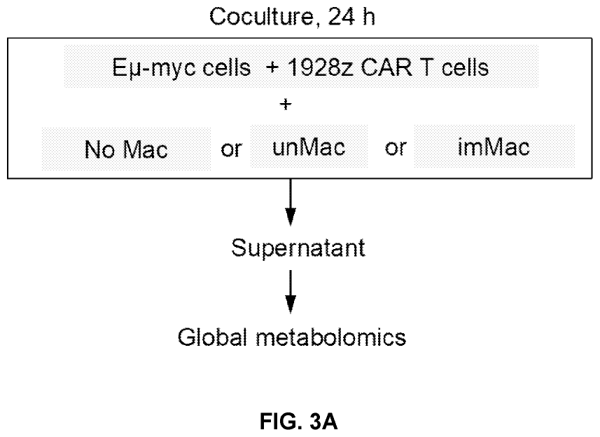

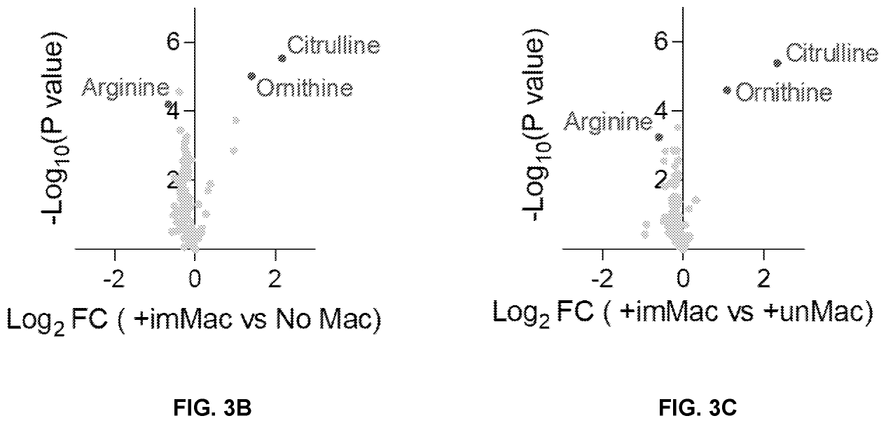

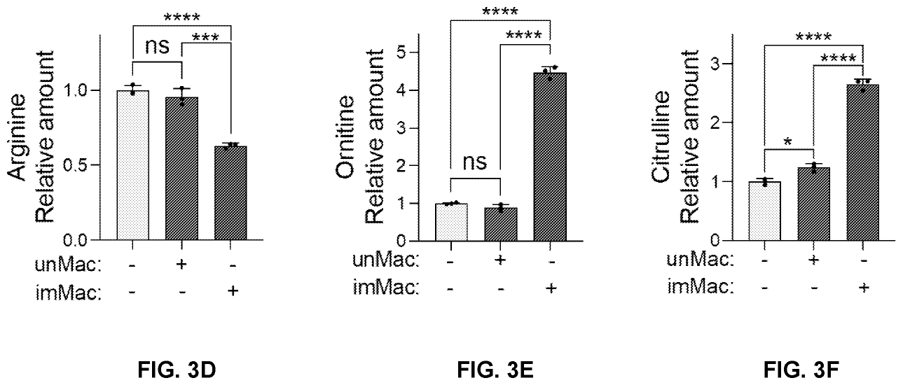

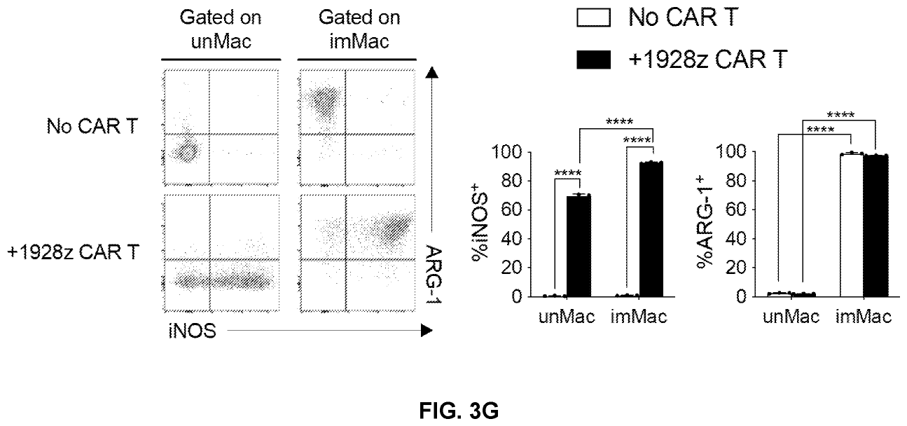

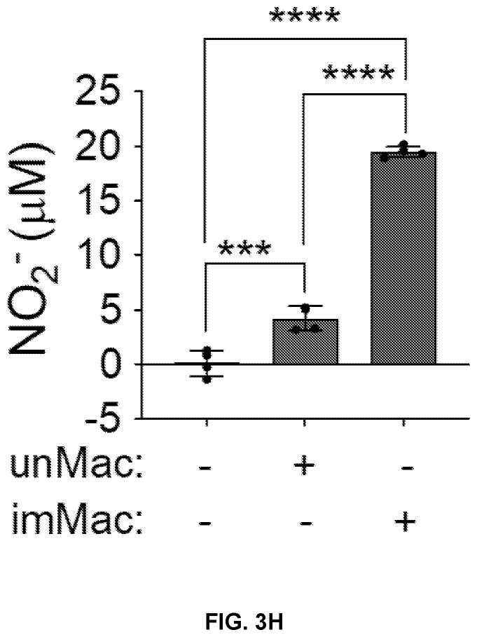

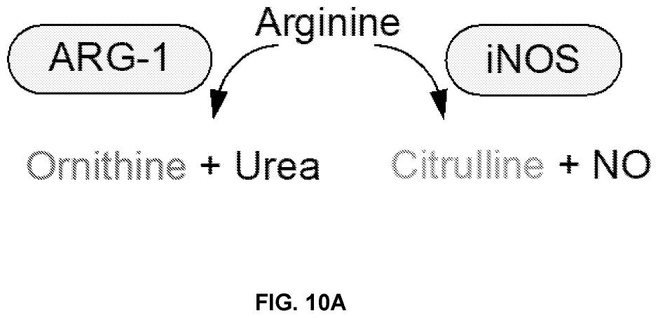

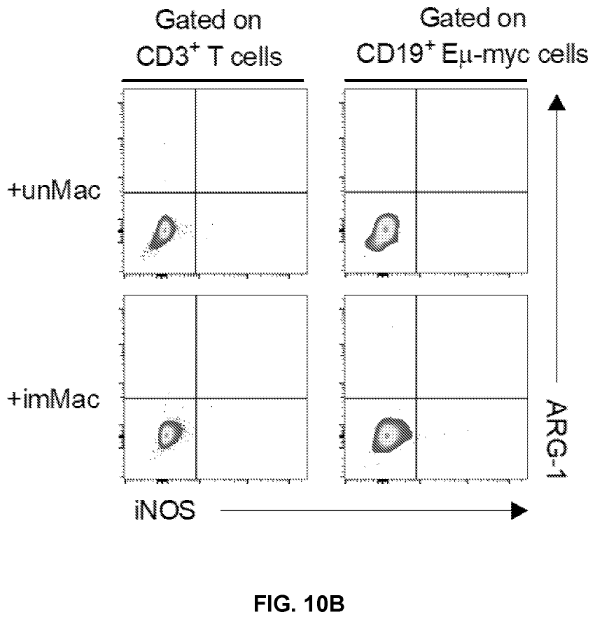

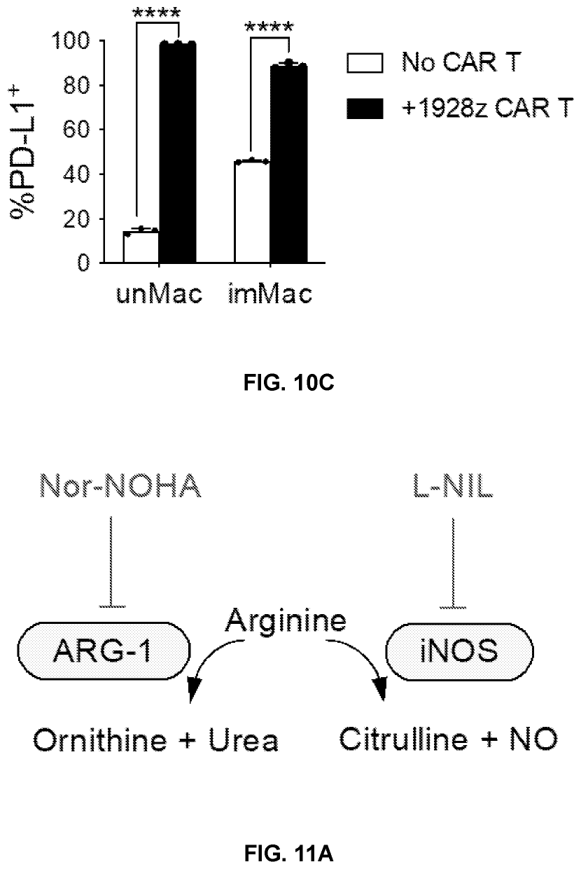

The metabolic crosstalk between CAR T cells and imMac was next interrogated to investigate the potential involvement of immune-metabolic alterations. A comprehensive analysis of metabolite profiles was conducted in the supernatants collected from the coculture model via global metabolomics using liquid chromatography-mass spectrometry (LC-MS) (FIG. 3A). There was a significant increase in citrulline and ornithine levels and a concomitant reduction in arginine levels within the supernatants derived from cocultures containing imMac compared to cocultures containing unMac or no macrophages (No Mac) (FIG. 3B-3F). Macrophages possess the capacity to metabolize arginine through arginase-1 (ARG-1) or inducible nitric oxide synthase (iNOS) pathways, producing ornithine and urea or citrulline and nitric oxide (NO), respectively (Bronte, et al. Nat Rev Immunol 2005 5:641-654) (FIG. 10A). In the absence of CAR T cells, imMac exhibited high expression levels of ARG-1 but minimal expression of iNOS, whereas unMac displayed minimal expression of both ARG-1 and iNOS (FIG. 3G). When CAR T cells were cocultured with macrophages, there was a significant induction of iNOS expression in imMac and, to a lesser extent, in unMac. The expression level of ARG-1 in macrophages remained unchanged regardless of the presence of CAR T cells in the cocultures. Neither CD3⁺ T cells nor Eμ-myc cells expressed ARG-1 or iNOS, confirming that the expression of these enzymes was limited to macrophages in this model (FIG. 10B). Consistent with the enhanced iNOS expression in imMac, higher levels of NO were secreted in cocultures with imMac compared to cocultures with unMac or without macrophages (FIG. 3H). Additionally, coculture with CAR T cells substantially increased the expression of PD-L1 in both unMac and imMac (FIG. 10C). Together, these findings demonstrate that exposure of imMac to CAR T cells triggers immunoregulatory features, including enhanced arginine catabolism through the upregulation of iNOS.

iNOS Upregulation in imMac Drives Suppression of CAR T Cell Function

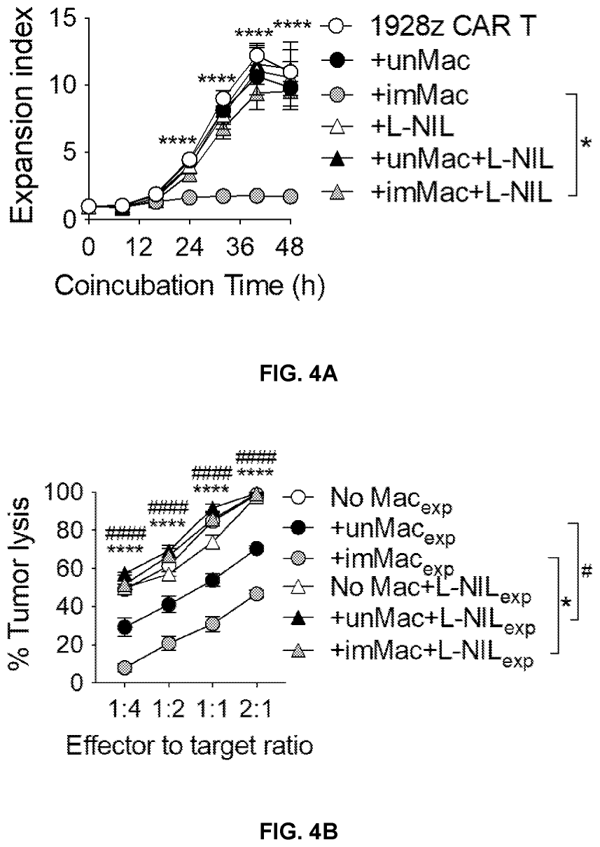

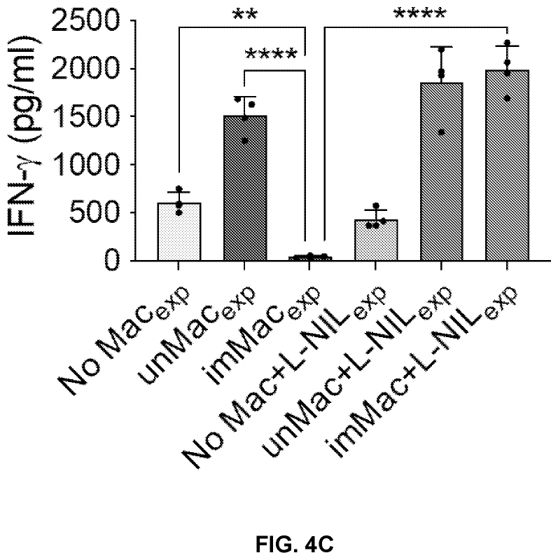

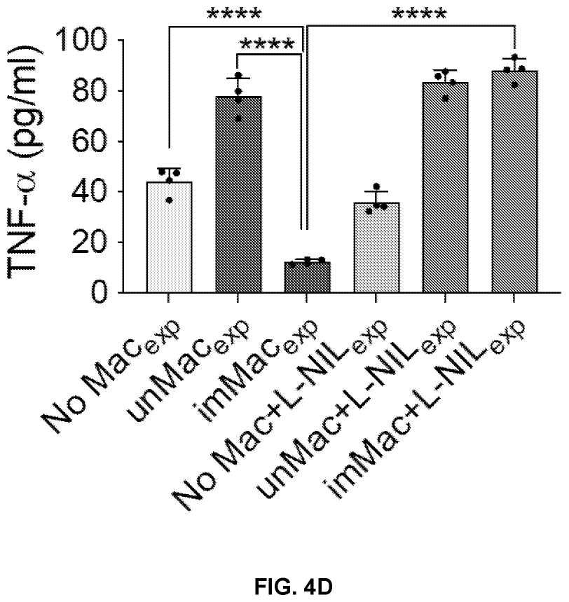

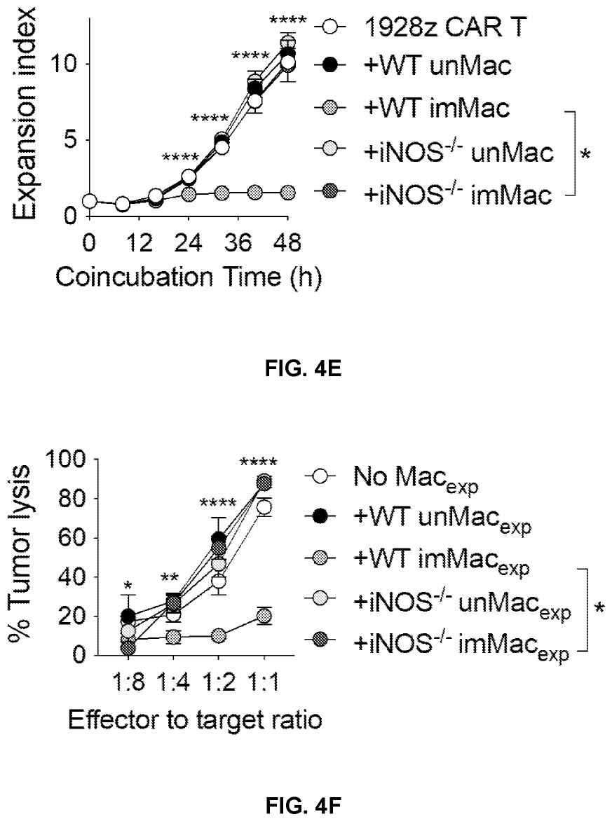

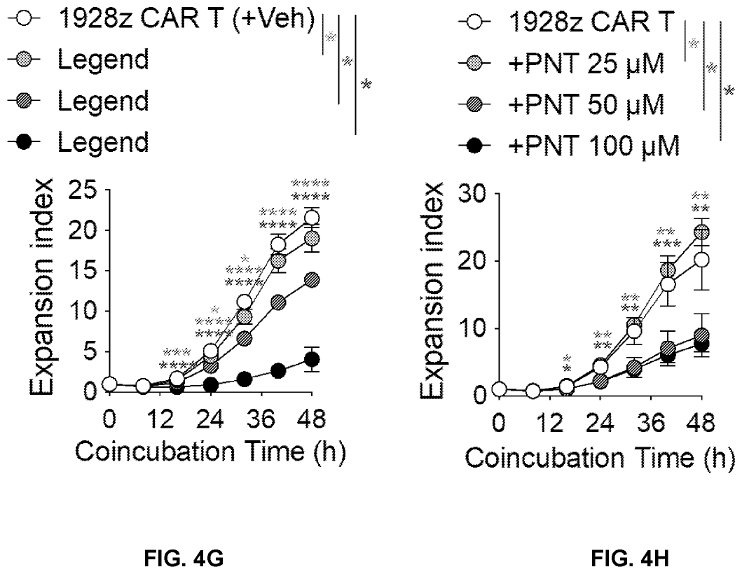

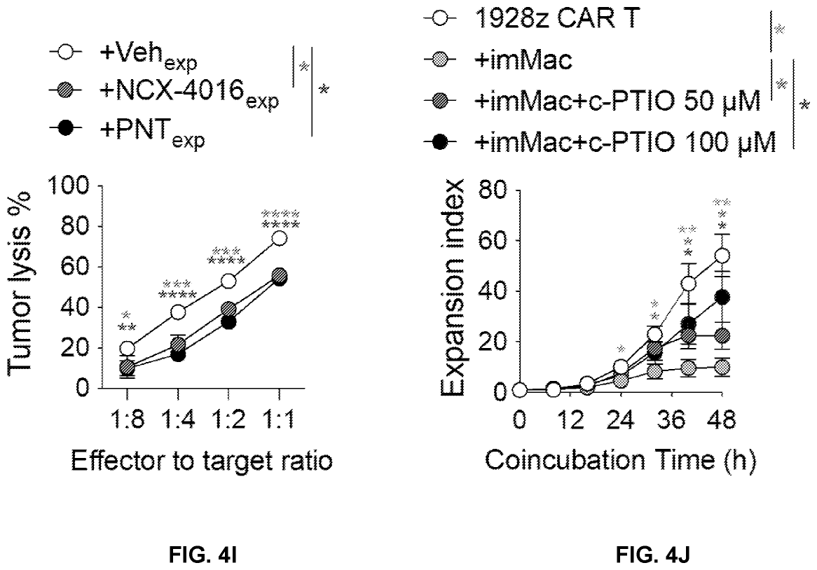

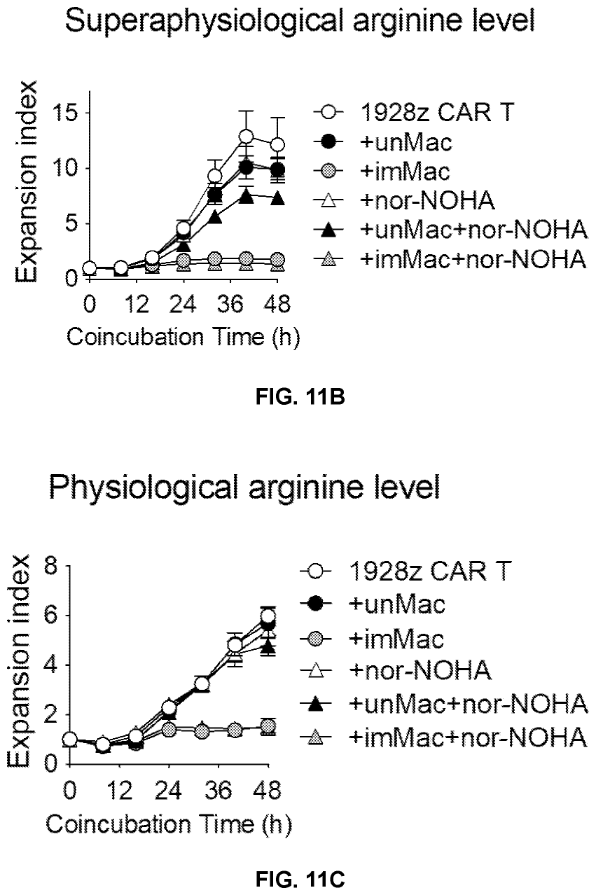

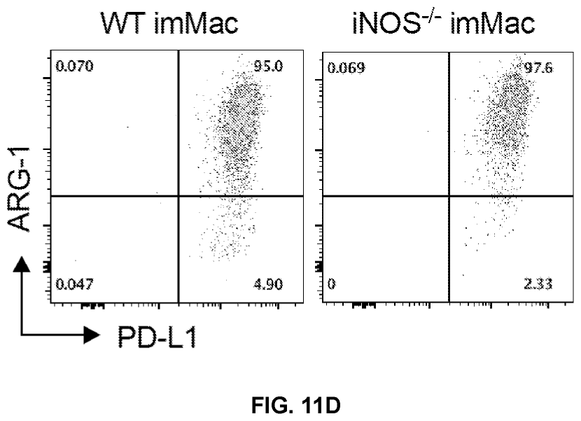

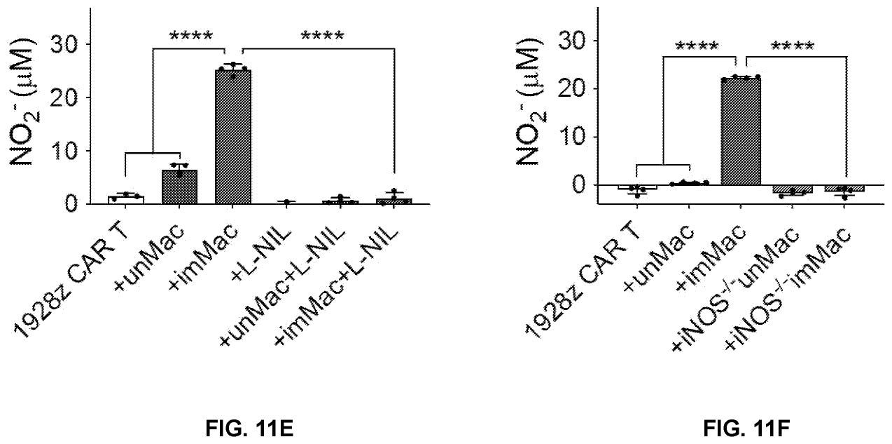

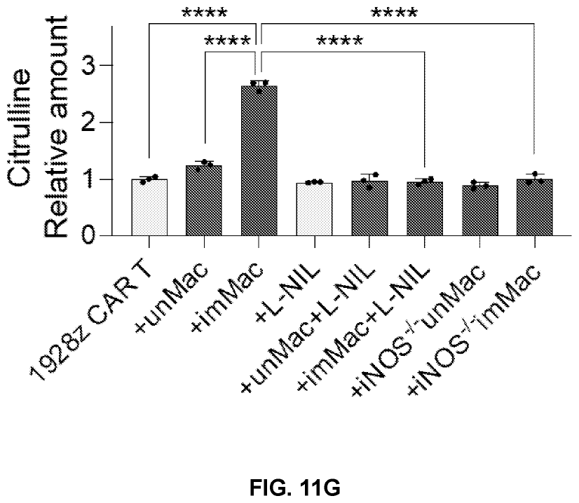

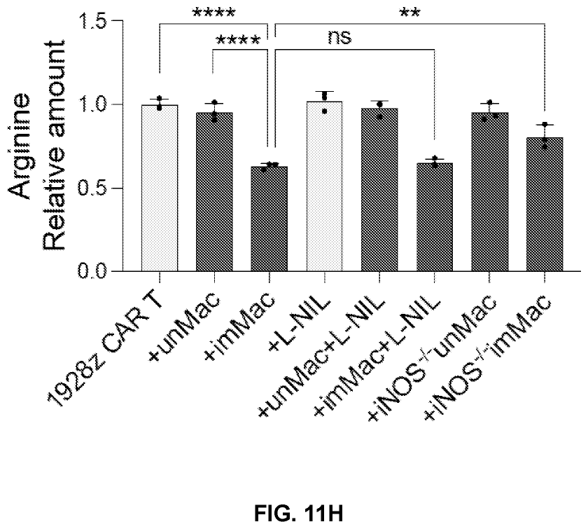

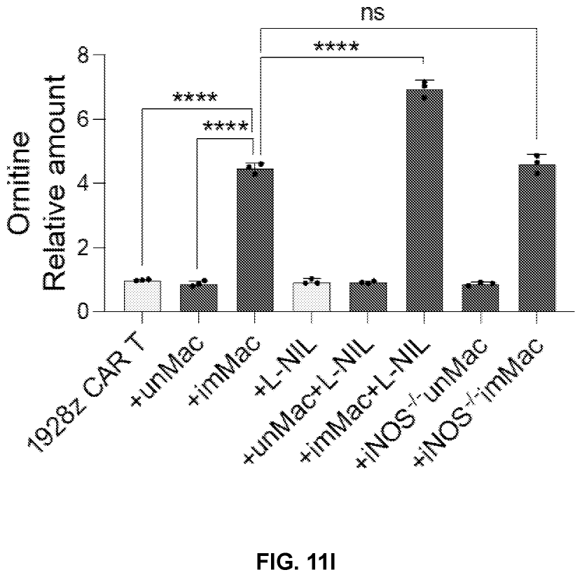

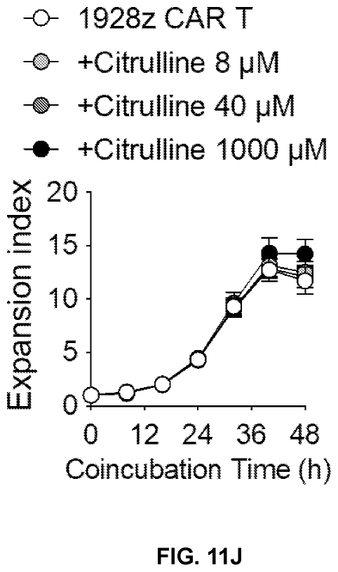

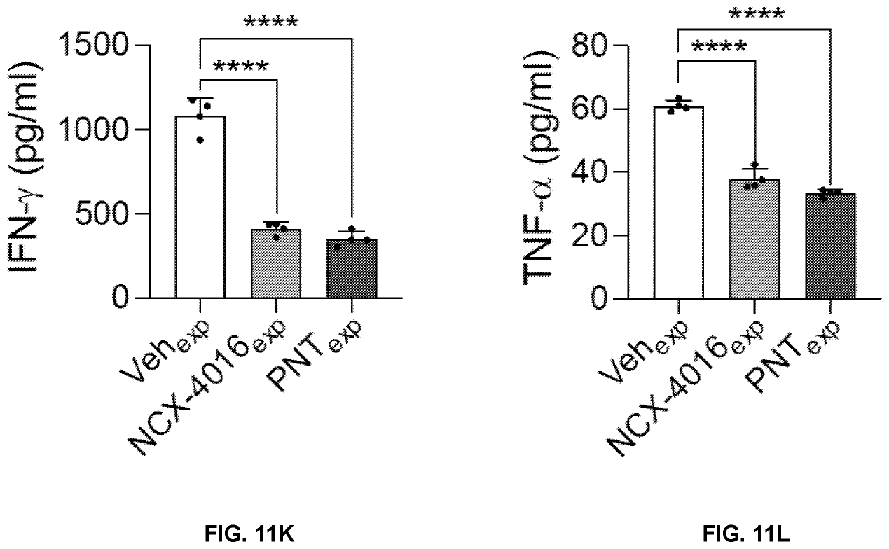

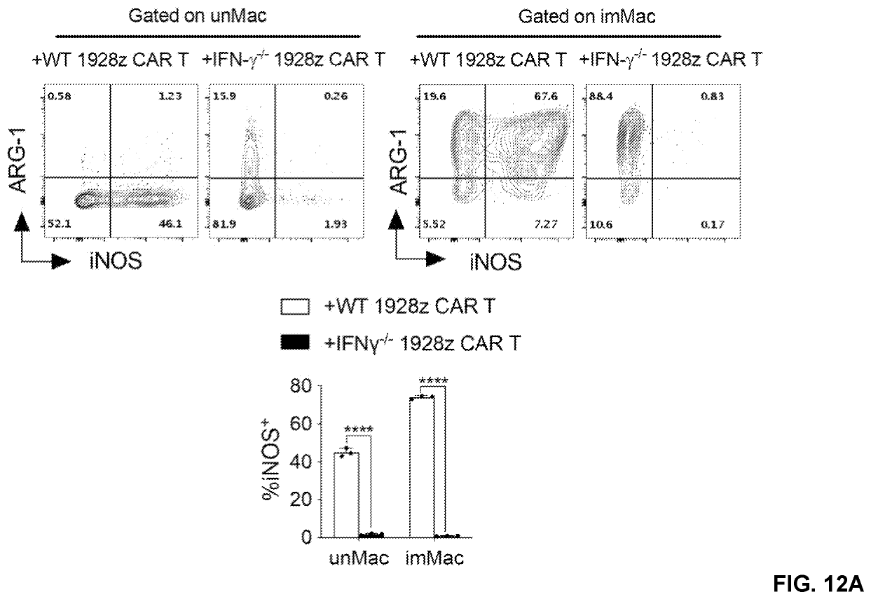

To investigate whether arginine metabolism by imMac contributes to the impairment of CAR T cell function, the effects of ARG-1 and iNOS inhibitors was examined (FIG. 11A). Treatment with the ARG-1 inhibitor nor-NOHA (Tenu et al., Nitric Oxide 1999 3:427-438) did not restore CAR T cell expansion in cocultures with imMac (FIG. 11B). The supraphysiological arginine concentration (1.15 mM) in culture media could diminish the effect of arginine depletion by ARG-1, thus the coculture was repeated at the physiological arginine concentration (150 μM) (Rodriguez, et al. Immunol Rev 2008 222:180-191). However, even in the context of physiological arginine levels, treatment with nor-NOHA failed to rescue CAR T cell expansion (FIG. 11C), suggesting ARG-1 by itself is not sufficient to inhibit CAR T cells. Next, the cocultures were treated with the iNOS inhibitor L-NIL (Giavridis et al., Nat Med 2018 24:731-738; Moore et al., J Med Chem 1994 37:3886-3888) showing a complete rescue of CAR T cell expansion in cocultures with imMac (FIG. 4A). Furthermore, L-NIL treatment preserved the capacity of CAR T cells to kill tumor cells (FIG. 4B) and produce effector cytokines (FIGS. 4C and D). Moreover, imMac developed from iNOS-deficient (iNOS) mice BMDMs did not inhibit CAR T cell expansion (FIG. 4E) or impair CAR T cell tumor killing capacity (FIG. 4F). Importantly, iNOS/-imMac expressed similar levels of ARG-1 and PD-L1 as wild-type (WT) imMac (FIG. 11D), indicating that these factors were not responsible for the suppression of CAR T cell function by imMac. Inhibition or genetic ablation of iNOS attenuated the production of NO (FIGS. 11E and 11F) and citrulline (FIG. 11G) in the cocultures with imMac. However, the levels of arginine and ornithine remained comparable regardless of iNOS ablation or inhibition (FIGS. 11H and 11I), ruling out their altered levels as the drivers of CAR T cell impairment. These findings collectively demonstrate that imMac suppresses CAR T cell function through the enzymatic activity of iNOS.

Next investigated was whether the iNOS products, citrulline and NO, were responsible for CAR T cell suppression by imMac. Exposure to high levels of citrulline did not impact expansion of CAR T cells (FIG. 11J). NO reacts with superoxide to form peroxynitrite (PNT, ONOO⁻), which leads to protein oxidation, lipid peroxidation, and DNA damage (Harris, et al. Trends Cell Biol 2020 30:440-451). Treatment with the NO-donor NCX-4016 or PNT resulted in the inhibition of CAR T cell expansion (FIGS. 4G and 4H) and diminished ability of CAR T cells to kill tumor cells (FIG. 41) and secrete effector cytokines IFN-γ and TNF-α (FIGS. 11K and 11L). Notably, treatment with NO-scavenger carboxyl-PTIO (c-PTIO) partially rescued expansion of CAR T cells during cocultures with imMac (FIG. 4J). These data indicate that NO and PNT act as key mediators of iNOS-induced dysfunction of CAR T cells.

CAR T Cell-Derived IFN-γ Induces iNOS in imMac

Secretion of cytokines, such as IFN-γ and TNF-α, by activated CAR T cells activates macrophages (Alizadeh et al., Cancer Discov 2021 11:2248-2265

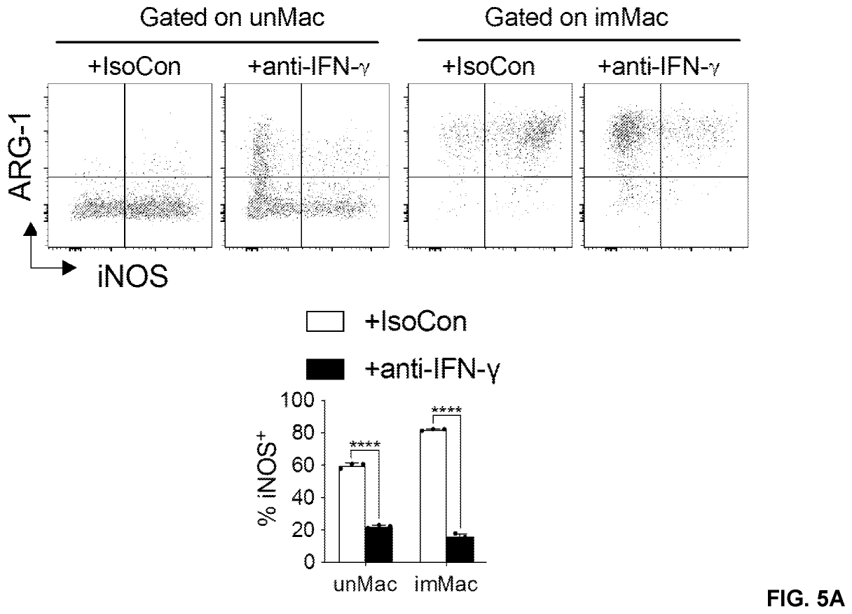

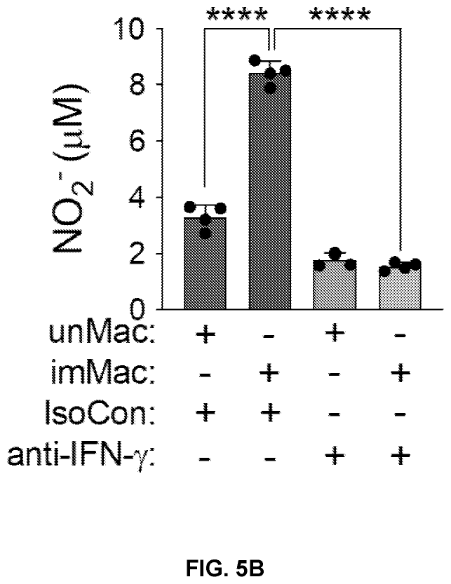

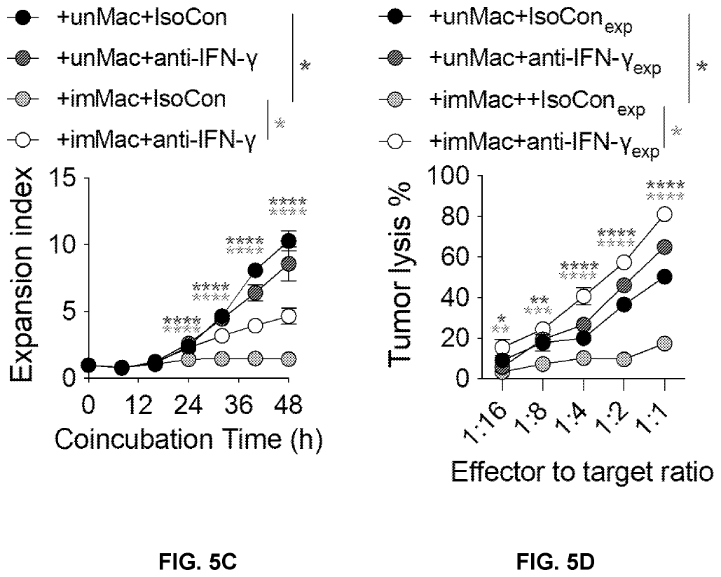

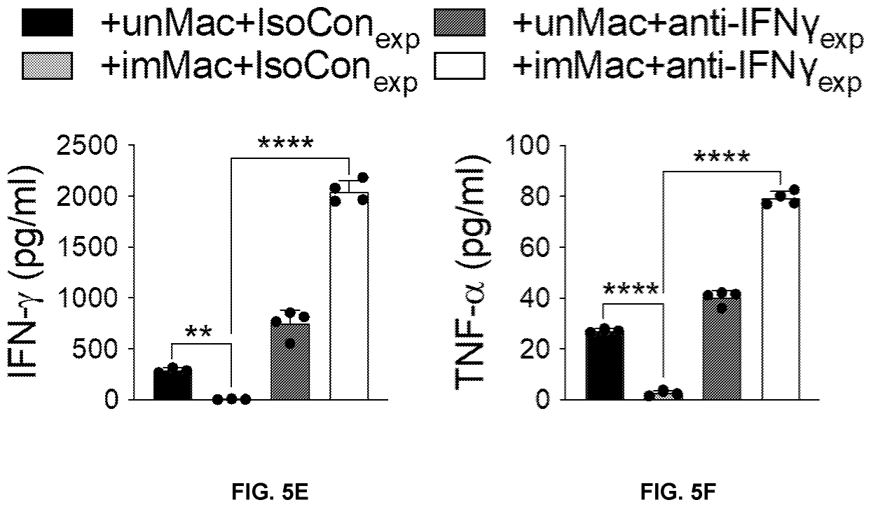

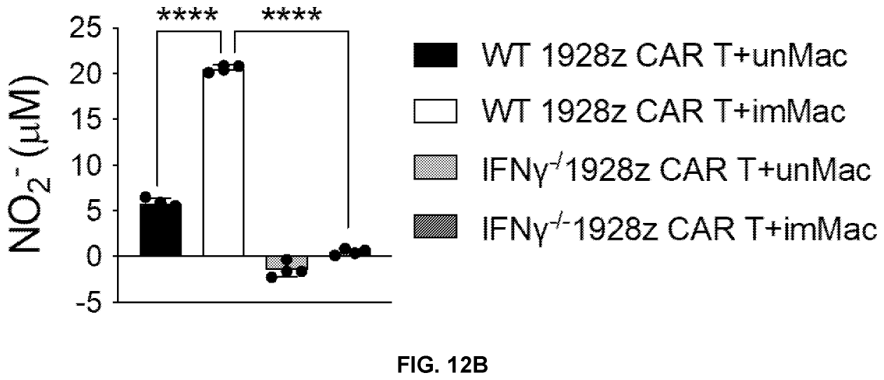

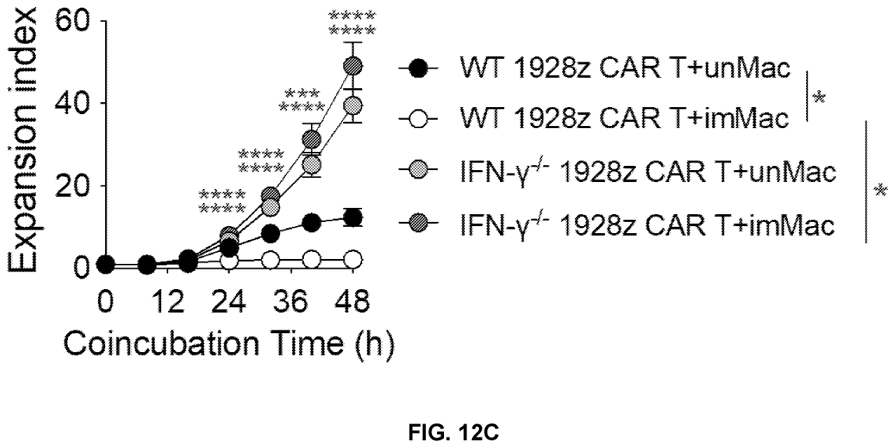

48. Bailey et al., Blood Cancer Discov 2022 3:136-153). A previous study reported that CAR T cell-derived IFN-γ upregulates iNOS in TAMs in a lung adenocarcinoma mouse model (Srivastava et al., Cancer Cell 2021 39:193-208). Neutralization of IFN-γ with blocking antibodies attenuated CAR T cell-triggered iNOS expression in unMac and imMac (FIG. 5A) and reduced the production of NO (FIG. 5B). In cocultures with imMac, treatment of anti-IFN-γ enhanced CAR T cell expansion (FIG. 5C) and preserved the ability of CAR T cells to lyse tumor cells (FIG. 5D) and produce IFN-γ and TNF-α (FIGS. 5E and 5F). Also, CAR T cells deficient in IFN-γ (IFN-γ/CAR T cells) neither induced iNOS expression in unMac and imMac (FIG. 12A) nor induced NO production in the cocultures (FIG. 12B). Moreover, IFN-γ^{-/-} CAR T cells exhibited enhanced expansion during cocultures with imMac (FIG. 12C). These findings demonstrate that blocking IFN-γ in CAR T cells blunts counter-regulatory iNOS-driven inhibitory activity of imMac.

iNOS-Expressing imMac Induces CAR T Cell Metabolic Dysregulation

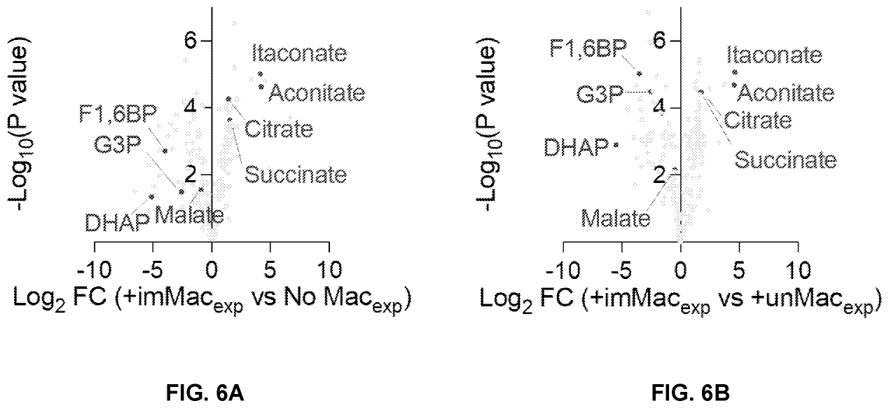

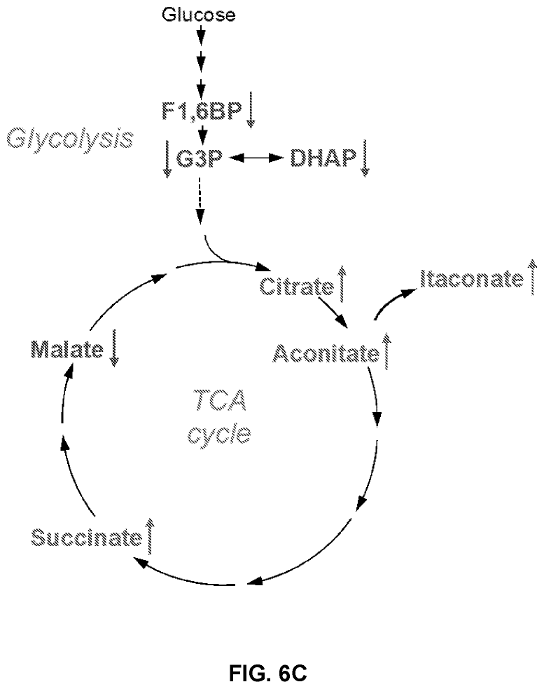

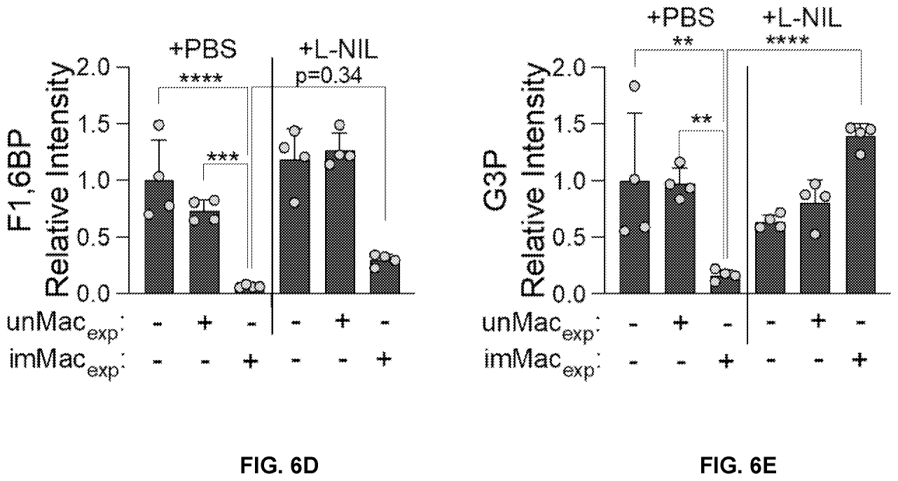

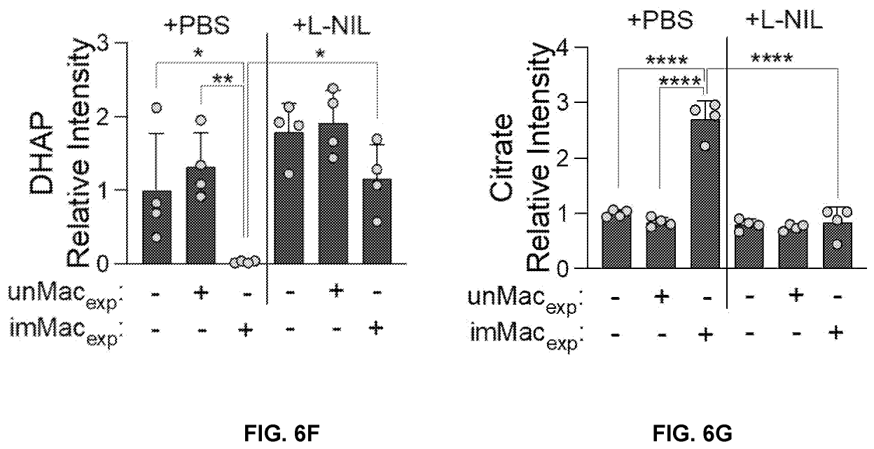

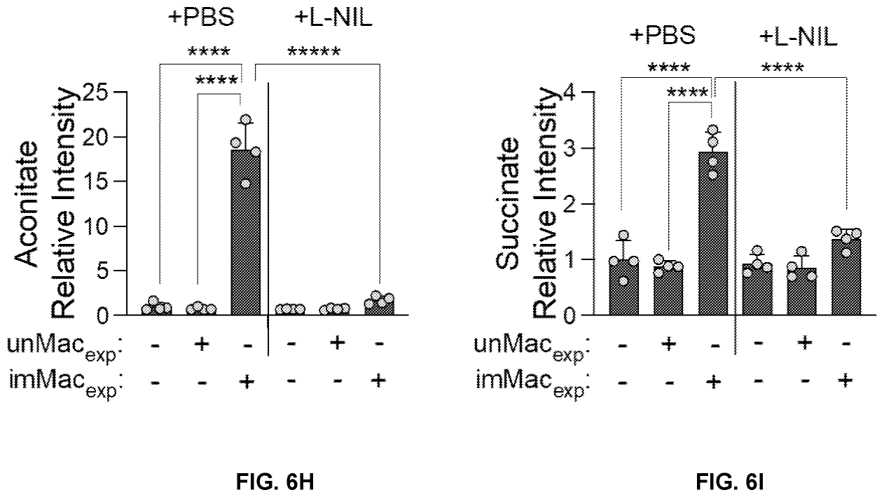

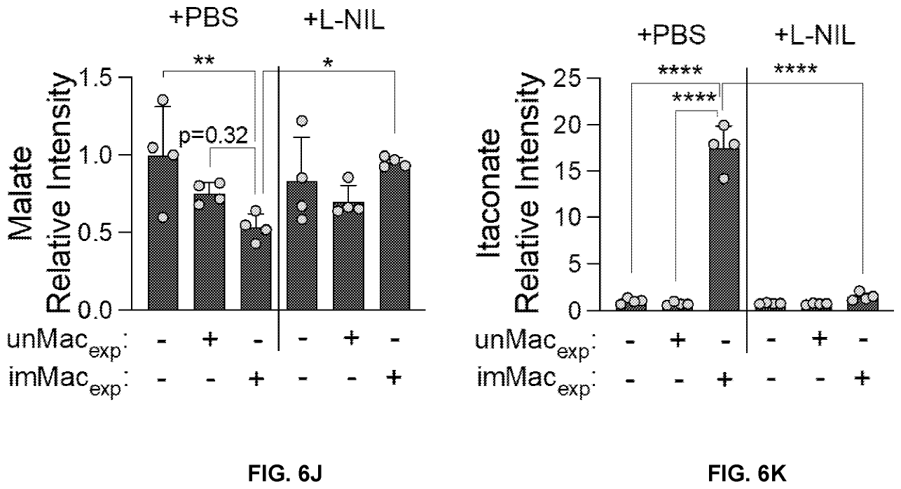

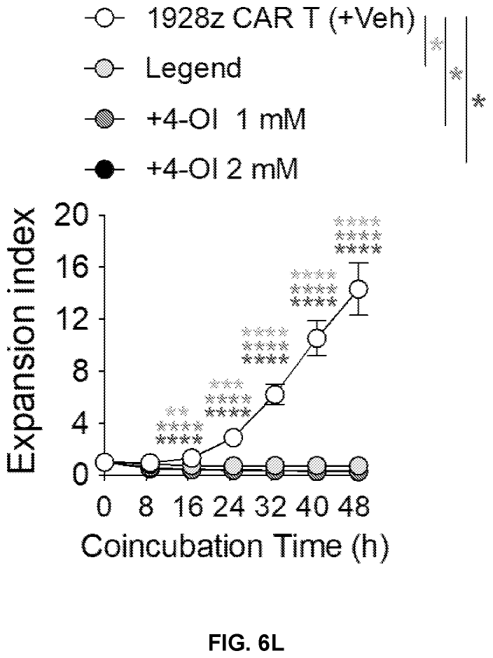

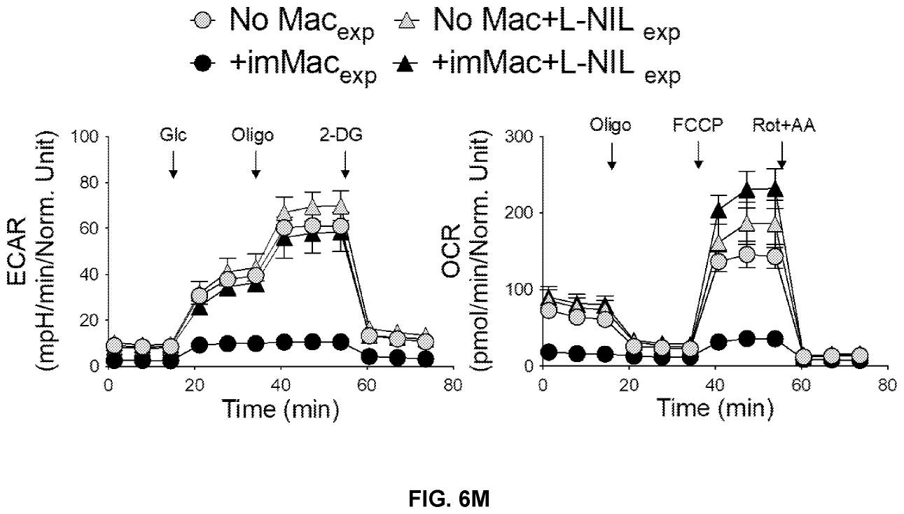

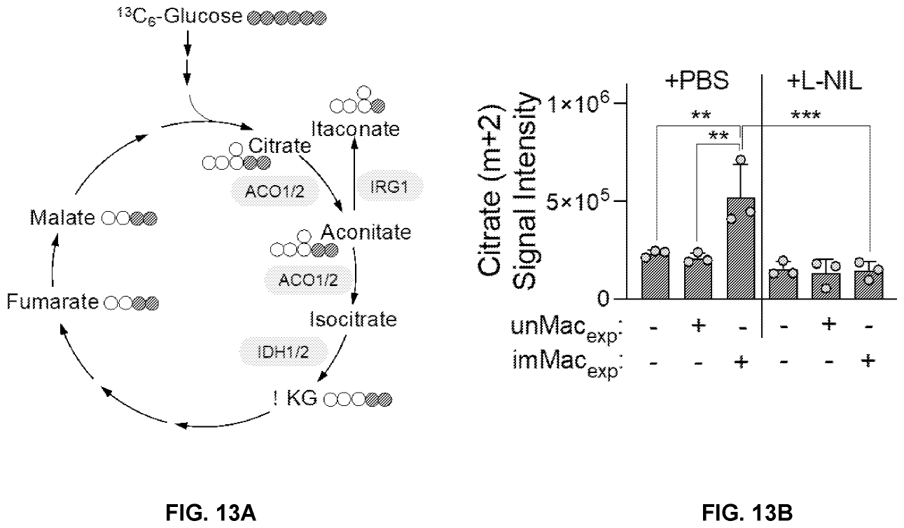

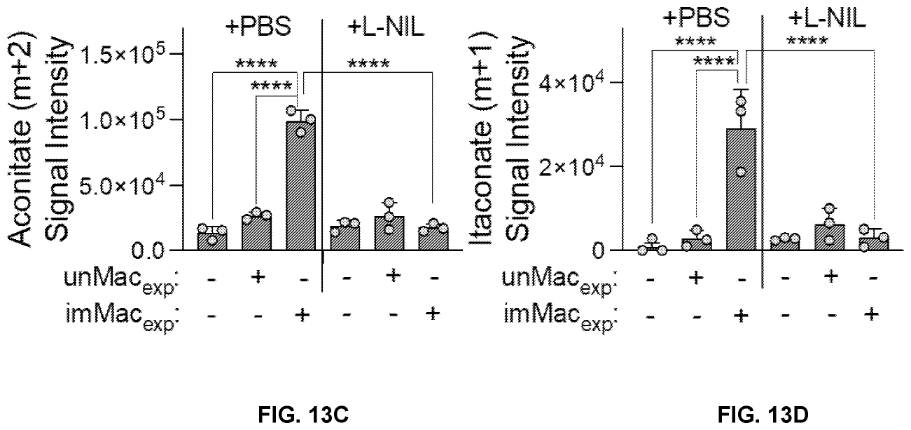

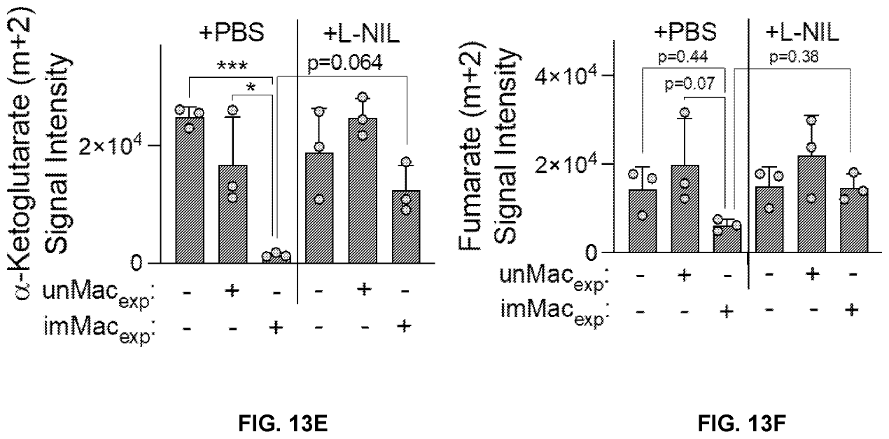

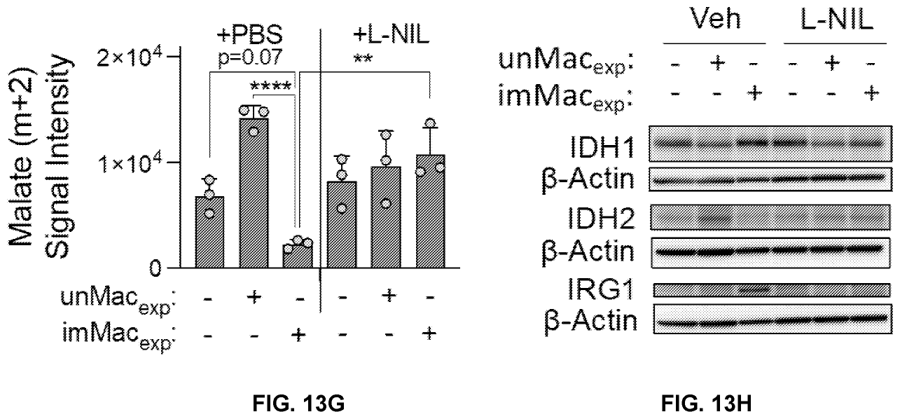

Cellular metabolism plays a crucial role in supporting the rapid proliferation and effector function of T cells (Bantug, et al. Nat Rev Immunol 2018 18:19-34; Reina-Campos, et al. Nat Rev Immunol 2021 21:718-738). To investigate the potential dysregulation of CAR T cell metabolism by imMac, global metabolomics analysis was conducted on CAR T cells from the coculture model. There were significant alterations in glycolytic and TCA cycle intermediates in CAR T cells cocultured with imMac (FIG. 6A-6C). Notably, the depletion of fructose 1,6-bisphosphate (F1,6BP), glyceraldehyde 3-phosphate (G3P), and dihydroxyacetone phosphate (DHAP) was found to be dependent on iNOS activity due to its reversal with L-NIL treatment (FIG. 6D-6F). Conversely, there was an iNOS-dependent accumulation of citrate, aconitate, and succinate, along with a decrease in malate (FIG. 6G-6J). There was also a substantial accumulation of itaconate in CAR T cells cocultured with imMac (FIG. 6K). Itaconate is synthesized from aconitate via immune response gene 1 (IRG1), also known as aconitate decarboxylase (ACOD1), in tumor-associated myeloid cells and suppresses the proliferation and cytolytic activity of CD8+ T cells (Zhao et al., Nat Metab 2022 4:1660-1673). Exposure to a cell-permeant form of itaconate, 4-octyl itaconate (4-OI) (Mills et al., Nature 2018 556:113-117), impaired CAR T cell expansion (FIG. 6L). Given the concurrent accumulation of itaconate with citrate and aconitate in CAR T cells cocultured with imMac, next investigated was whether CAR T cells can produce itaconate. Through ¹³C₆-glucose tracing on CAR T cells, iNOS-dependent accumulation of ¹³C-labeled citrate, aconitate, and itaconate, as well as a reduction of ¹³C₆-labeled α-ketoglutarate (αKG), fumarate, and malate was identified in CAR T cells cocultured with imMac (FIG. 13A-13G). Immunoblot analysis revealed the increased expression of IRG1 in CAR T cells cocultured with imMac, while the expression of isocitrate dehydrogenase 2 (IDH2) was decreased (FIG. 13H). These results indicate that imMac, via iNOS, induces metabolic vulnerability in CAR T cells by depleting glycolytic intermediates and rewiring the TCA cycle to divert aconitate towards itaconate production instead of αKG. Consistent with the altered metabolite profiles, extracellular flux demonstrated that CAR T cells from cocultures with imMac exhibited attenuated glycolytic and oxidative metabolic activities, as evidenced by decreased extracellular acidification rate (ECAR) and oxygen consumption rate (OCR), which are proxies for glycolytic rate and mitochondrial oxidative phosphorylation, respectively (FIG. 6M). Importantly, L-NIL treatment preserved glycolytic and oxidative metabolic capabilities of CAR T cells in the presence of imMac. These findings demonstrate the broad disruption of CAR T cell metabolic programs by imMac, underscoring the importance of targeting iNOS to sustain the metabolic fitness of CAR T cells.

iNOS Inhibition Improves CAR T Cell Therapeutic Efficacy

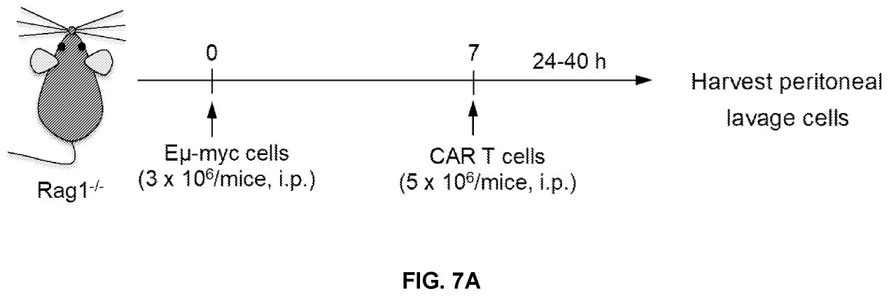

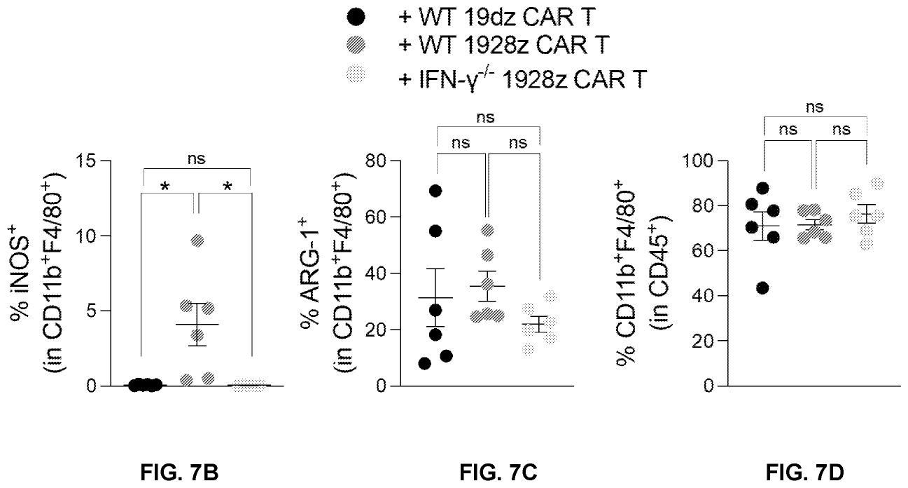

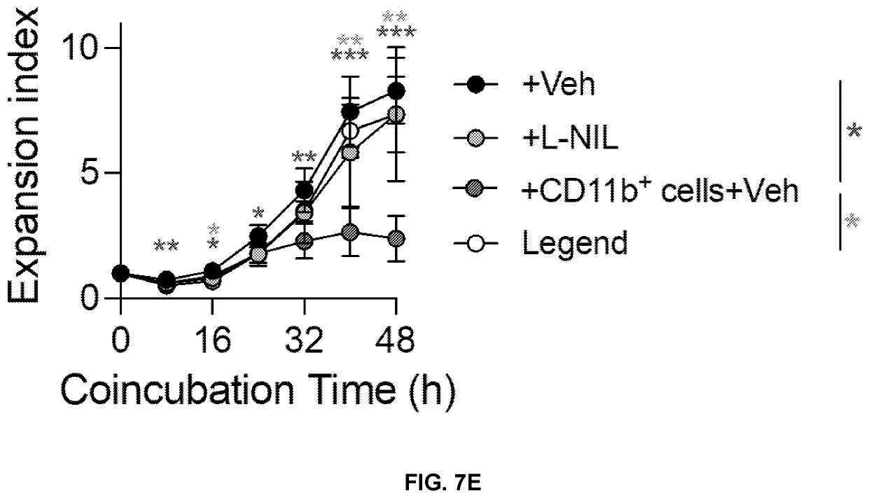

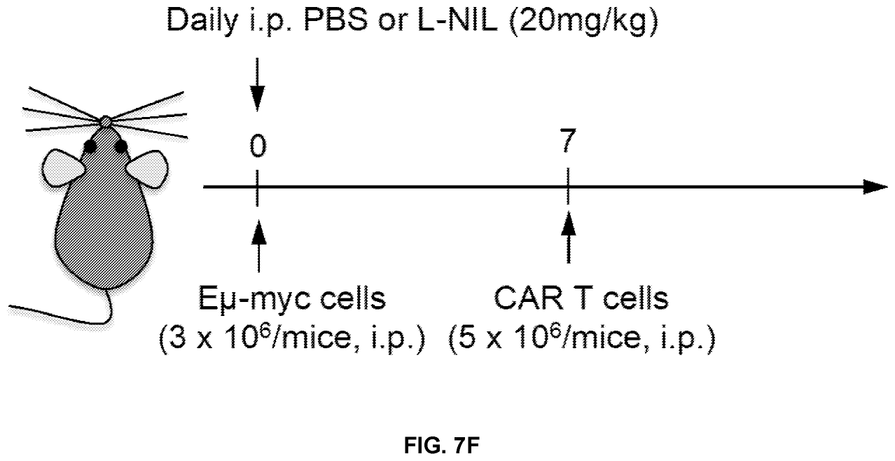

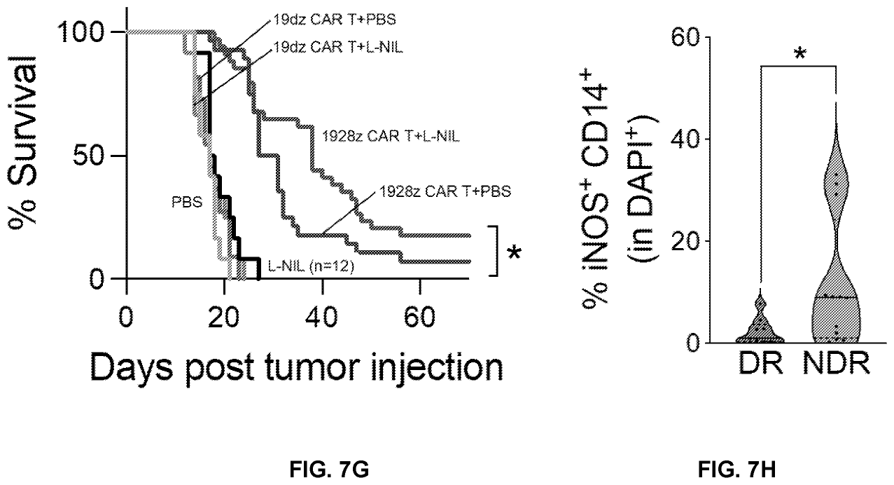

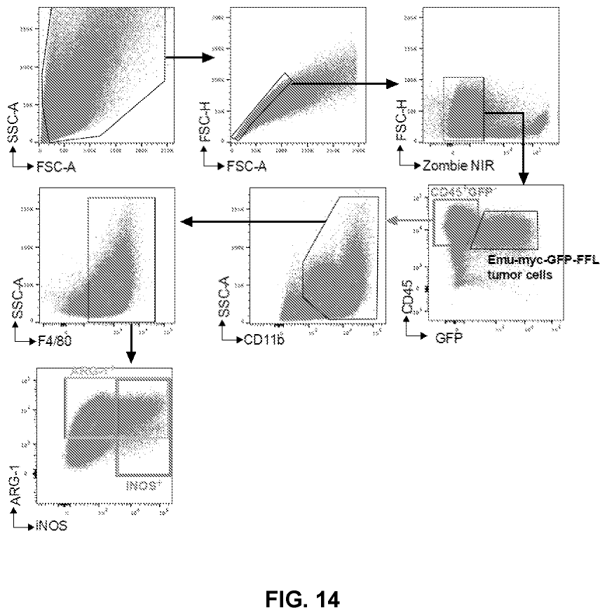

Next investigate was whether iNOS limits the effectiveness of CAR T cell therapy in vivo. To control the source of IFN-γ production, Rag1/-mice, which lack endogenous T and B cells, were utilized. Eμ-myc B cell tumors were established in the peritoneal cavity (FIG. 7A). CAR T cells carrying a non-functional control CAR (19dz) that contains a truncated CD3ζ were used as controls (FIG. 9). Intraperitoneal transfer of WT 1928z, IFN-γ^{-/-} 1928z, or WT 19dz CAR T cells was performed to facilitate direct interaction with macrophages at the tumor site. The frequency of iNOS+F4/80+ macrophages was significantly increased in WT 1928z CAR T cell-treated mice compared to mice treated with IFN-γ^{-/-} 1928z or WT 19dz CAR T cells (FIGS. 7B and 14A). The frequencies of ARG1⁺ F4/80+ macrophages and total CD11b⁺F4/80⁺ macrophages were similar across all groups (FIGS. 7C and 7D). Furthermore, CD11b⁺ myeloid cells from the peritoneal cavity of WT 1928z CAR T cell-treated tumor-bearing mice suppressed expansion of fresh antigen-naïve CAR T cells ex vivo in an iNOS-dependent manner (FIG. 7E). Next, assessed was whether inhibition of iNOS could improve therapeutic efficacy of CAR T cells (FIG. 7F). Mice treated with a combination of 1928z CAR T cells and L-NIL exhibited significantly improved survival compared to mice treated with 1928z CAR T cells alone (FIG. 7G).

These results demonstrate that IFN-γ produced by CAR T cells stimulates iNOS in macrophages at the tumor site, and concurrent inhibition of iNOS enhances the therapeutic activity of CAR T cells. While iNOS expression in macrophages has been attributed to induction of the CRS after CAR T cell treatment (Giavridis et al., Nat Med 2018 24:731-738), it has not been noted to be associated with therapeutic resistance in patients. Therefore, to investigate if iNOS expression correlates to clinical outcomes in patients receiving CD19-targeted CAR T cells, the expression of iNOS in CD14⁺ myeloid cells was evaluated within pre-infusion patient tumors. Multiplex immunofluorescence analysis on tumor tissue microarrays (FIG. 1C) confirmed a higher proportion of iNOS+CD14+ cells in patients with NDR compared to the patients with DR (FIG. 7H). These data provide further support that iNOS expression within the TME contributes to non-durable responses to CAR T cell therapy in LBCL patients.

### DISCUSSION

These findings indicate that the upregulation of iNOS in imMac, provoked by IFN-y secreted by CAR T cells, impairs various aspects of CAR T cell biology, including expansion, effector function, and metabolic capacity, all of which can reduce the therapeutic efficacy of CAR T cells. Consistent with observations, increased expression of iNOS in various cancers has been associated with poor prognosis, highlighting the strong immunosuppressive effects of iNOS (Chen, et al. J Surg Oncol 2006 94:226-233; Eyler et al., Cell 2011 146:53-66; Glynn et al J Clin Invest 2010 120:3843-3854; Granados-Principal et al., Breast Cancer Res 2015 17:25). These data show that, despite the expression of iNOS in unMac, they do not exert suppressive effects on CAR T cells. This could be attributed to the lower extent of iNOS upregulation and NO production in unMac compared to imMac. Moreover, under conditions of low arginine levels, iNOS catalyzes the production of superoxide, leading to the formation of PNT (Nathan, et al. Nat Rev Immunol 2013 13:349-361). This may, in part, explain the significant suppression of CAR T cells by iNOS⁺ARG-1⁺ imMac compared to iNOS+ARG-1⁻ unMac. The molecular mechanisms underlying this suppression remain to be elucidated. Further investigations into the transcriptional and translational regulation of CAR T cells by iNOS-expressing imMac are warranted. Nevertheless, the capacity of CAR T cells to stimulate iNOS in host macrophages emphasizes the potential for CAR T cell treatment regimens that regulate iNOS derived from myeloid cells in TME.

CAR T cell dysfunction induced by iNOS involves repression of glycolytic and oxidative metabolic activity. The metabolic profiles of CAR T cells have been shown to be crucial for their antitumor activity, persistence, and differentiation into memory T cells (Fraietta et al., Nat Med 2018 24:563-571; Kawalekar et al., Immunity 2016 44:380-390). Thus, the contribution of TME-associated myeloid-derived factors in shaping CAR T cell metabolism might determine CAR T cell behavior and treatment outcomes. This study unveils, for the first time, a rewiring of the TCA cycle in CAR T cells, resulting in the accumulation of itaconate triggered by iNOS-expressing imMac. Notably, the expression of IRG1 and the production of itaconate were previously identified in macrophages and MDSCs, but not in T or natural killer (NK) cells, as immunosuppressive mediators (Zhao et al., Nat Metab 2022 4:1660-1673; Chen et al., Sci Adv 2023 9: eadg0654). The mechanisms underlying the stabilization of IRG1 in CAR T cells and the extent to which endogenous production of itaconate contributes to CAR T cell dysfunction is an important future research direction. Understanding the regulation of CAR T cell metabolism within TME and developing strategies to sustain their metabolic fitness will enhance the efficacy and durability of CAR T cell therapy.

This work highlights the role of IFN-γ as an initiator of the iNOS-dependent inhibitory circuit between CAR T cells and imMac. Blocking IFN-γ in CAR T cells effectively eliminates the suppressive effects mediated by imMac. The role of IFN-γ in the TME is paradoxical, as it can promote tumor cell apoptosis but can also limit antitumor immunity by upregulation of inhibitory molecules, such as PD-L1, PD-L2, indoleamine 2,3-dioxygenase 1 (IDO), FAS, and FAS ligand (FASL) (Gocher, et al. Nat Rev Immunol 2022 22:158-172). Similarly, CAR T cell-derived IFN-γ has been shown to activate host antitumor immunity and sustain CAR T cell cytotoxicity (Alizadeh et al., Cancer Discov 2021 11:2248-2265; Boulch et al., Sci Immunol 2021 6) but can also induce macrophage production of cytokines and chemokines associated with CRS (Bailey et al., Blood Cancer Discov 2022 3:136-153). Neutralizing IFN-γ mitigated CAR T therapy-associated toxicity without compromising the antitumor efficacy of CAR T cells against lymphoma and leukemia (Bailey et al., Blood Cancer Discov 2022 3:136-153; Manni et al., Nat Commun 2023 14:3423). Moreover, the tumor cytotoxic effects of CAR T cell-derived IFN-γ are dependent on the intrinsic sensitivity of cancer cells to IFN-γ signaling (Larson et al., Nature 2022 604:563-570; Boulch et al., Nat Cancer 2023). Therefore, the impact of IFN-γ on the TME and tumor progression is determined by various factors, including immune cell composition, cellular phenotypes, and tumor genetics. Further investigations are needed to determine the extent to which targeting IFN-γ can enhance CAR T therapy in a tumor type-specific manner.

There is a higher abundance of M2-like TAMs in the pre-CAR T cell treatment TME of NDR patients. A high density of M2-like TAMs within the tumor stroma has been associated with poor prognosis in several cancer types due to their tolerogenic immunoregulatory potential, in contrast to M1-like TAMs that promote anti-tumor effects (Bruni, et al. Nat Rev Cancer 2020 20:662-680; Fridman, et al. Nat Rev Clin Oncol 2017 14:717-734). These finding also suggests that the suppressive TAM phenotype has a significant impact on clinical responses and may contribute to the local immunosuppression observed in non-responder TMEs (Scholler et al., Nat Med 2022 28:1872-1882). Additionally, circulating monocytic MDSCs (M-MDSCs) can migrate to the TME and differentiate into TAMs (Kumar et al., Immunity 2016 44:303-315; Veglia, et al. Nat Immunol 2018 19:108-119; Kumar, et al. Trends Immunol 2016 37:208-220), potentially contributing to the inferior responses observed in patients with elevated numbers of peripheral blood M-MDSCs (Jain et al., Blood 2021 137:2621-2633). Therefore, developing biomarkers to identify the macrophage phenotypes present within the TME of individual patients, and to identify those at high-risk for poor outcomes, will be crucial for translating the findings into improved clinical outcomes. Pre-clinical and clinical data suggest that these patients can be identified by elevated pre-treatment levels of serum inflammatory markers IL-6, c-reactive protein (CRP), and Ferritin (Jain et al., Blood 2021 137:2621-2633; Faramand et al., Clin Cancer Res 2020 26:4823-4831). Therefore, future research is needed to better understand the relationship between TAMs and IL-6, CRP, and Ferritin to develop interventions that overcome suppressive macrophages within the TME. These efforts will increase the probability of achieving more frequent durable responses from CAR T cell therapy.

### Materials and Methods

### Patient Samples

All samples were prospectively obtained from patients with relapsed or refractory LBCL who underwent axi-cel treatment at H. Lee Moffitt Cancer Center. The collection of samples was conducted in accordance with approved protocols by the institutional review board. Pre-treatment tumor biopsies were obtained within 1 month prior to axi-cel infusion and before lymphodepletion. Patients who achieved sustained remission for at least 6 months following axi-cel infusion were classified as durable responders (DR). Non-durable responders (NDR) were patients who either experienced lymphoma relapse or passed away due to any cause.

### Mice

All animal studies were performed according to a protocol approved by H. Lee Moffitt Cancer Center and Research Institute and the University of South Florida Institutional Animal Care and Use Committee. C57BL/6J mice, Nos2^{-/-} (B6.129P2-Nos2^{tm1Lau}/J) mice, Ifng^{-/-} (B6.129S7-Ifng^{tm1Ts}/J) mice, and Rag1^{-/-} mice (B6.129S7-Rag1^{tm1Mom}/J) were purchased from Jackson Laboratories. Rag1^{-/-} mice were bred in-house.

### Cell Lines

Eμ-myc cells were derived from the axillary lymph node of tumor-bearing Eμ-myc transgenic mice, a spontaneous Burkitt-like lymphoma model (Davila, et al. PLOS One 2013 8: e61338; Adams et al., Nature 1985 318:533-538; Harris et al., J Exp Med 1988 167:353-371). For some experiments, Eμ-myc cells that were retrovirally transduced to express GFP-firefly luciferase (Eμ-myc-GFP-FFL) were used. Eμ-myc cells were maintained on irradiated (30 Gy) NIH-3T3 feeder cells in RPMI-1640/IMDM (1/1, v/v) supplemented with 10% heat-inactivated fetal bovine serum (HI-FBS), 2 mM L-glutamine, 100 U/ml Penicillin/Streptomycin, and 22.5 UM B-mercaptoethanol. Prior to use as feeder cells, NIH/3T3 was maintained in DMEM supplemented with 10% HI-FBS, 2 mM L-glutamine, and 100 U/ml Penicillin/Streptomycin. Cell lines were routinely tested for the absence of mycoplasma contamination using the Universal Mycoplasma Detection kit (ATCC) or MycoAlert PLUS mycoplasma detection kit (Lonza).

### Genomics

RNA-sequencing was performed as previously described (Scholler et al., Nat Med 2022 28:1872-1882; Faramand et al., Clin Cancer Res 2020 26:4823-4831). Formalin-fixed paraffin-embedded (FFPE) or snap-frozen samples were obtained and examined by a hematologist for tumor content. RNA was extracted and RNA-sequencing libraries were prepared using NuGen RNA-Seq Multiplex System (Tecan US) according to the manufacturer's protocols. The libraries were then sequenced on the Illumina NextSeq 500 system with a 75-base paired-end run at 80 to 100 million read pairs per sample. To determine the immune cell composition in bulk RNA-seq profiles of tumor biopsies, CIBERSORTx v.1.0.41 was applied with the LM22 signature matrix. Geneset enrichment analysis of M2-associated gene expression was performed on the R package GSVA, utilizing a panel of genes as previously described (Martinez, et al. J Immunol 2006 177:7303-7311).

### Multiplex Immunofluorescence

Multiplex immunofluorescence staining was performed as previously described (Jain et al., Blood 2021 137:2621-2633). FFPE tumor biopsies were obtained and examined by a hematopathologist for tumor content. Tissue microarray (TMA) consisting of 21 cores was created from a total of 11 patients (9 cores from 6 patients with DR, 12 cores from 5 patients with NDR). The TMA was then subjected to staining by OPAL multiplexing method, based on Tyramide Signal Amplification (TSA) on the Leica BOND® Automated Stainer (Leica Biosystems, Wetzlar, Germany), using two sets of antibodies against specific markers: 1) CD68 (CST, D4B9C, HIER-Citrate pH 6.0, 1:100, dye520), PAX5 (Abcam, EPR3730 (2), HIER-EDTA PH 9.0, 1:400, dye690), CD4 (Cell Marque, EP204, HIER-EDTA PH 9.0, 1:100, dye570), CD163 (Abcam, OTI2G12, HIER-Citrate pH 6.0, 1:50, dye540), FOXP3 (Abcam, 236A/E7, HIER-EDTA PH 9.0, 1:200, dye620), and CD3 (Thermo Fisher, SP7, HIER-EDTA PH 9.0, 1:400, dye650). After the final stripping step, DAPI counterstain is applied to the multiplexed slide and is removed from BOND RX for coverslipping with ProLong Diamond Antifade Mountant (ThermoFisher Scientific). One additional Fluorescent Multiplex-IF panels were designed using there following antibodies: 2) iNOS (Thermo Fisher, 4E5, HIER-EDTA PH 9.0, 1:100, dye620) and CD14 (Abcam, LPSR/2386, HIER-EDTA pH 9.0, 1:300, dye570). All slides were imaged with the Vectra®3 Automated Quantitative Pathology Imaging System. Images were analyzed using HALO Image Analysis Platform (Indica Labs, Albuquerque, NM).

### Generation of Retroviral Constructs

Plasmids encoding 19dz and 1928z CAR constructs in SFG γ-retroviral vectors have been described previously (Boucher et al., Cancer Immunol Res 2021 9:62-74). Briefly, 1928z CAR construct includes anti-murine CD19 scFv (1D3), murine CD8a transmembrane and hinge domains, murine CD28 intracellular domain, and murine CD3z intracellular domain followed by the mCherry reporter via glycine-serine linker. 19dz CAR construct includes the same sequence as 1928z construct except for absence of CD28 intracellular domain and having a truncated CD3z intracellular domain. For retrovirus production, plasmids were transfected to H29 cell lines using a calcium phosphate transfection kit (Invitrogen) to produce vesicular stomatitis virus G-glycoprotein-pseudotyped retroviral supernatants. These retroviral supernatants were subsequently transduced to Phoenix-ECO cell lines to stably generate Moloney murine leukemia virus-pseudotyped retroviral particles.

### Mouse T Cell Isolation and CAR T Cell Generation

Mouse spleens were excised, mechanically disrupted, and filtered through a 40 μm cell strainer.

CD3⁺ T cells were enriched via negative selection using EasySep Mouse T Cell Isolation Kit (STEMCELL Technologies). T cells were activated and expanded with anti-CD3/28 Dynabeads (Gibco) at a bead-to-cell ratio of 0.8:1. T cells were spinoculated (2000×g, 1 h, room temperature) twice, 24 h and 48 h after initial T cell activation, with viral supernatants collected from Phoenix-ECO cells on retronectin (Takara) coated plates. Following the second spinoculation, T cells were maintained for one day. On day 5, anti-CD3/28 Dynabeads were removed, and CAR T cells were used for in vitro or in vivo experiments. CAR transduction efficiency was determined by flow cytometry as a percentage of mCherry⁺ cells in live cells. Mouse T cells were cultured in RPMI-1640 supplemented with 10% HI-FBS, 2 mM L-glutamine, 100 U/ml Penicillin/Streptomycin, 1× nonessential amino acids, 1 mM sodium pyruvate, 10 mM HEPES, 55 μM 2-mercaptoethanol, and 100 IU/ml recombinant human IL-2.

### Animal Experiment

Six-to 10-week-old Rag1/-mice of both sexes were intraperitoneally (i.p.) injected with 3×10⁶ Eμ-myc-GFP-FFL cells to generate tumors localized in peritoneal cavity. Tumor engraftment was verified by bioluminescence imaging one day before CAR T cell transfer. Mice were randomized to different treatment groups without differences in pre-treatment tumor load. Seven days after tumor cell inoculation, mice were injected i.p. with 5×10⁶ CAR T cells in 300 μl PBS. For survival experiments, L-NIL or PBS was administered i.p. once per day at 20 mg/kg body weight starting on the same day of tumor cell injection. Experimental endpoints were achieved when mice demonstrated signs of morbidity or hind-limb paralysis, or when solid tumor masses reached 2000 mm³ for some mice that developed palpable masses. Bioluminescence imaging was performed by IVIS Lumina III In Vivo Imaging System (PerkinElmer) with Living Image software (PerkinElmer).

### Macrophage Development and Polarization

BMDMs were generated from bone marrow cells harvested from femurs and tibias of WT or iNOS^{-/-} mice. Following red blood cell lysis by ACK (Ammonium-Chloride-Potassium) lysis buffer, 1×10⁷ bone marrow cells were cultured in 10-cm tissue culture dish in 10 ml of RPMI-1640 supplemented with 10% HI-FBS, 2 mM L-glutamine, 100 U/ml Penicillin/Streptomycin, and 20 ng/ml M-CSF (R&D systems) for 7 days. On day 3, 10 ml of fresh medium with 20 ng/ml M-CSF was added. On day 5, the culture medium was entirely discarded and replaced by 15 ml of fresh medium with 20 ng/ml M-CSF. On day 6, BMDMs were activated for 24 h with 20 ng/ml of IL-4 and IL-10 (Peprotech) (77, 78) to develop imMac or cultured in media only to use as unMac. M-CSF (20 ng/ml) was added during activation with cytokines. On day 7, adherent cells were harvested by gentle scraping and used for in vitro experiments.

### Mouse Peritoneal Cells

Peritoneal cells were obtained by peritoneal lavage as previously described (Giavridis et al., Nat Med 2018 24:731-738). After euthanizing mice, 5 ml ice-cold PBS/2 mM EDTA were i.p. injected. Bellies were massaged for one minute, and subsequently incised to drain the lavage fluid in a collection tube. Cells were filtered through a 40 μm cell strainer. Following red blood cell lysis with ACK lysing buffer, peritoneal cells were used for subsequent analyses. For ex vivo coculture experiments with CAR T cells, EasySep Mouse CD11b Positive Selection Kit II (STEMCELL Technologies) was used to isolate CD11b⁺ myeloid cells.

### Expansion Assay

The expansion of CAR T cells was determined by an Incucyte S3 live cell analysis system (Essen Bioscience). 2×10⁴ Eμ-myc cells and 2×10⁴ CAR T cells were cocultured in the absence or presence of 0.5×10⁴ macrophages (CAR T:Eμ-myc cell:Macrophage=1:1:0.25, unless otherwise indicated in the figure legends) in a 96-well black-walled clear bottom plate in 120 μl of media. Cell images were captured at 4× magnification. The expansion index was calculated by dividing the total integrated red intensity (RCU×μm²/mm²) at each time point by the first time point.

### Griess Assay

2×10⁴ Eμ-myc cells and 2×10⁴ CAR T cells were cocultured in the presence or absence of 0.5×10⁴ macrophages in a 96-well plate in 120 μl of media. Coculture supernatants were harvested, and nitric oxide levels were measured using Griess reagent system (Promega) according to manufacturer's instructions. Absorbance was read at 560 nm using microplate reader (GloMax, Promega), and NO₂-concentrations were determined by standard curve. Standard curve was prepared with diluting 0.1M sodium nitrite standard (provided in the kit) with the culture media used for experiments.

### BrdU Incorporation Assay

2×10⁵ Eμ-myc cells and 2×10⁵ CAR T cells were cocultured in the absence or presence of 0.5×10⁵ macrophages in a 24-well plate in 1200 μl of media. At 24 h of coculture, BrdU was added to each well at 10 UM. After an additional incubation for 18 h, cells were harvested. BrdU staining was performed according to APC BrdU flow kit (BD Pharmingen) and BrdU incorporation was analyzed by flow cytometry.

### Flow Cytometry

The following fluorophore-conjugated anti-mouse antibodies were used. From BD Horizon: anti-CD45 (30-F11), anti-CD19 (1D3), anti-CD11b (M1/70), and anti-CD38 (145-2C11). From BioLegend: anti-CD8a (53-6.7), anti-PD-L1 (10F.9G2), and anti-F4/80 (BM8). From eBioscience: anti-mouse ARG-1 (A1exF5) and anti-NOS2 (CXNFT). Fc receptors were blocked using FcR Blocking Reagent (anti-mouse CD16/CD32 antibody, Invitrogen). DAPI (BD Pharmingen) and Zombie NIR Fixable Viability Kit (BioLegend) were used as viability dyes. For intracellular staining, surface-labeled cells were fixed and permeabilized with Cytofix/Cytoperm kit (BD Biosciences) according to the manufacturer's instructions and then stained with intracellular antibodies. For cell surface CAR staining, protein L-biotin conjugate followed by PE-conjugated streptavidin was used. Flow cytometry was performed on a LSR II or FACSymphony instrument (BD Biosciences). Data were analyzed with the FlowJo software (FlowJo LLC).

CAR T Cell Isolation from Initial Coculture for Subsequent Downstream Assays

1×10⁶ CAR T cells and 1×10⁶ Eμ-myc-GFP-FFL cells were cocultured in the absence or presence of 0.25×10⁶ macrophages in a 6-well plate in 6 ml of media for 48 h. After initial coculture, cells were harvested, and T cells were isolated using Mouse T Cell Isolation Kit (STEMCELL Technologies). T cell purity was 100% as tested by flow cytometry. Percentage of CAR-expressing T cells was determined with flow cytometry and were subsequently used for downstream assays.

### Luciferase-Based Killing Assay

2×10⁴ Eu-ALL-GFP-FFL cells were cocultured with CAR T cells at different effector-to-target ratios in a 96-well white-walled plate in 100 μl of media. Following incubation, 100 μl luciferase substrate reagent (ONE-Glo Luciferase assay system, Promega) was added to each well. Target cells alone were plated at the same cell density to determine maximum luciferase signals. Emitted luminescence was detected in the microplate reader (GloMax, Promega). Percent lysis was determined as (1-sample signal/maximum signal)×100.

### Cytokine Secretion Assay

2×10⁴ Eμ-myc cells were cocultured with 2×10⁴ CAR T cells in a 96-well plate in a total volume of 100 μl of media. Supernatants were collected and analyzed for IFN-γ and TNF-α secretion using Ella automated immunoassay system (Proteinsimple Bio-techne) according to manufacturer's instructions.

### Immunoblotting

Cells were lysed in ELB lysis buffer (50 mM HEPES, PH 7.5, 250 mM NaCl, 5 mM EDTA, 0.5 mM DTT, 0.1% NP-40 alternative, 10 μg/ml aprotinin, 10 μg/ml leupeptin, and 100 μg/ml trypsin/chymotrypsin inhibitor). Following protein quantification with the Pierce BCA protein assay (ThermoFisher), the samples were mixed with a loading buffer containing 2-mercaptoethanol. The proteins were electrophoresed in 4-20% Tris-Glycine gels (Novex-Invitrogen) and transferred to PVDF membrane with a Bio-Rad Trans-Blot SD Semi-Dry Transfer Cell. The membrane was blocked in 5% bovine serum albumin (BSA) in TBST and subsequently blotted with primary and secondary antibodies in 5% BSA in TBST. The following antibodies were used: IDH1 (clone D2H1; Cell signaling, #8137S), IDH2 (clone D2E3B; Cell signaling, #56439S), IRG1 (clone E5B2G; Cell signaling, #19857S), B-actin (clone AC-74, Sigma-Aldrich, #A2228), and horseradish peroxidase-conjugated secondary antibodies (Donkey-anti-Rabbit, Cytiva, #NA934-1ML); Sheep-anti-Mouse, Cytiva, #NA931-1ML). Membranes were imaged with a ChemiDoc Imaging System (BioRad, #17001401) and exported through ImageLab (Bio-Rad #12012931).

Metabolomics and ¹³C₆-Labeled Glucose Tracing Analyses

For global metabolomics analysis of cell-cultured medium, the cell-free medium was obtained by performing rapid centrifugation (17,000×g, 10 sec, room temperature) to collect the supernatant. The metabolites present in 20 μl of the cell-cultured medium were then extracted using 80 μl of ice-cold MeOH. Following a 30 min incubation on ice and subsequent centrifugation (17,000×g, 20 min, 4° C.), the supernatant was subjected to LC-HRMS analysis.

Global metabolomic profiling and 13C6-labeled glucose tracing of CAR T cells, 1×10⁶ T cells were resuspended in either RPMI-1640 medium (RPMI+10% heat-inactivated dialyzed FBS) or 13C6-glucose substituted RPMI-1640 medium (glucose-free RPMI+10% heat-inactivated dialyzed FBS+11.1 mM ¹³C₆-glucose). After 4 h incubation, cells were collected, rapidly centrifuged (17,000×g, 10 sec, room temperature), and medium was removed. T cells were washed with 1 ml of ice-cold PBS, and metabolites were extracted with 300 μl of 80% methanol via incubation at −80° C. for 15 min. Samples were centrifuged (17,000×g, 20 min, 4° C.), and supernatants were transferred to an Eppendorf tube and dried in a vacuum evaporator overnight. The dried extracts were resuspended in 20 μl of aqueous 50% methanol, clarified by centrifugation (17,000×g, 20 min, room temperature), and analyzed by LC-HRMS.

LC-HRMS analysis was performed on a Vanquish UPLC coupled with a Q-Exactive HF mass spectrometer, employing the same conditions as the previously established methods (Kang et al., Cell Metab 2021 33:174-189). A ZIC-PHILIC LC column (4.6 mm inner diameter×150 mm length, 5 μm particle size, MilliporeSigma, Burlington, MA) with a ZIC-PHILIC guard column (4.6 mm inner diameter×20 mm length, MilliporeSigma, Burlington, MA) was used for chromatographic separation at a column temperature of 30° C. The mobile phases consisted of 10 mM (NH₄)₂CO₃ and 0.05% NH₄OH in H₂O for mobile phase A, and 100% can for acetonitrile (ACN) mobile phase B. The LC gradient conditions were as follows: 0 to 13 min: a decreasing of 80% to 20% of mobile phase B, 13 to 15 min: 20% of mobile phase B. The ionization was set to negative mode, with the MS scan range set to 60 to 1000 m/z. The mass resolution was 70,000, and the AGC target was 1×10⁶. The sample loading volume was 5 μl.

The unlabeled or ¹³C-labeled metabolite peaks were extracted using EL-Maven with a metabolite standard-based in-house library. For global metabolomic profiling, peak areas of metabolites were normalized by the median value of the total for identified metabolite peak areas in each sample. For the ¹³C-labeled metabolite peaks, the natural isotope peak area was corrected using IsoCor (Version 2.2) (Millard et al., Bioinformatics 2019 35:4484-4487).

### Seahorse Assay

ECAR and OCR were measured using a Seahorse Extracellular Flux Analyzer (Agilent Technologies). XF96 microplates were coated with CellTak a day before analyses. To assay glycolytic function, T cells were resuspended in glucose-free XF medium supplied with 2 mM L-glutamine and 1 mM sodium pyruvate and seeded at 2×10⁵ cells in 180 μl per well. Following incubation in a CO₂-free incubator for 60 min at 37° C. for pH stabilization, ECAR was measured in response to 10 mM glucose, 1 μM oligomycin, and 50 mM 2-deoxyglucose. To assay mitochondrial function, T cells were resuspended in XF medium supplied with 2 mM L-glutamine, 1 mM sodium pyruvate, and 10 mM glucose and seeded at 2×10⁵ cells in 180 μl per well. Following incubation in a CO₂-free incubator for 60 min at 37° C. for pH stabilization, OCR was measured in response to 1 μM oligomycin, 1 μM FCCP, and 0.5 UM rotenone and antimycin.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for enhancing anti-tumor efficacy of immune effector cells in a subject, comprising administering to the subject an effective amount of a composition comprising a nitric oxide synthase (NOS) inhibitor, interferon gamma (IFN-γ) inhibitor, or combination thereof.

2. The method of claim 1, wherein the NOS inhibitor is a NOS2-specific inhibitor.

3. The method of claim 1, wherein the NOS inhibitor is a non-selective NOS inhibitor.

4. The method of claim 1, wherein the NOS inhibitor comprises at least one member selected from the group consisting of N-[4-(2-{[(3-chlorophenyl)methyl]amino}ethyl)phenyl]-2-thiophenecarboximide dihydrochloride, 7-nitroindazole, 1-(2-trifluoromethylphenyl) imidazole, [N5-(1-imino-3-butenyl)-L-ornithine], 3-bromo-7-nitroindazole, and S-ethyl-N-[4-(trifluoromethyl)phenyl) isothiourea HCl.

5. The method of claim 1, wherein the IFN-γ inhibitor is an IFN-γ neutralizing antibody.

6. The method of claim 1, wherein the IFN-γ inhibitor is a gene silencing oligonucleotide.

7. The method of claim 1, wherein the immune effector cell is selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell.

8. The method of claim 1, wherein the immune effector cell is a CAR-T cell.

9. The method of claim 1, wherein the immune effector cell is a tumor infiltrating lymphocyte (TIL).

10. The method of claim 1, wherein the immune effector cell is engineered to express the NOS inhibitor or IFN-γ inhibitor.
